SEQUENCE LISTING (10 sequences)

SEQ ID NO: 1 - protein, 9 aa, Artificial Sequence
  PHFP 1
ALDDAVNPL

SEQ ID NO: 2 - protein, 9 aa, Artificial Sequence
  PHFP 2
WQFNLLTHV

SEQ ID NO: 3 - protein, 9 aa, Artificial Sequence
  PHFP 3
TLQPITLEL

SEQ ID NO: 4 - protein, 9 aa, Artificial Sequence
  PHFP 4
RVIEVLHGL

SEQ ID NO: 5 - protein, 9 aa, Artificial Sequence
  PHFP 5
QLLGDVQRV

SEQ ID NO: 6 - protein, 9 aa, Artificial Sequence
  PHFP 6
NLLTHVESL

SEQ ID NO: 7 - protein, 9 aa, Artificial Sequence
  PHFP 7
FQINEQVLA

SEQ ID NO: 8 - protein, 9 aa, Artificial Sequence
  PHFP 8
SQHQWQFNL

SEQ ID NO: 9 - protein, 9 aa, Artificial Sequence
  PHFP 9
LQDEVTHRM

SEQ ID NO: 10 - protein, 9 aa, Artificial Sequence
  PHFP 10
YTVKFYDGV

(19) United States
(12) Patent Application Publication
(10) Pub. No.: US 2016/0017011 A1
(43) Pub. Date: Jan. 21, 2016

(54) PHF20 AND JMJD3 COMPOSITIONS AND METHODS OF USE IN CANCER IMMUNOTHERAPY

(71) Applicants: Rongfu WANG, (US); Yichen WANG, (US); Wei ZHAO, (US)

(72) Inventors: Rongfu WANG, Houston, TX (US); Yichen WANG, Houston, TX (US); Wei ZHAO, Houston, TX (US)

(21) Appl. No.: 14/770,467

(22) PCT Filed: Feb. 26, 2014
(86) PCT No.: PCT/US14/18464

§ 371 Date: Aug. 26, 2015

Related U.S. Application Data
(60) Provisional application No. 61/769,545, filed on Feb. 26, 2013.

(51) Int. Cl. C07K 14/47; A61K 35/15; G01N 33/569; C07K 16/18; C12N 15/113; A61K 39/395; A61K 38/08

(52) U.S. Cl. CPC C07K 14/4702; A61K 39/3955; A61K 35/15; A61K 38/08; C07K 16/18; C12N 15/1138; G01N 33/56977; C07K 2317/24; C07K 2317/21; C07K 2317/76; C12N 2310/14; C12N 2320/30; G01N 2500/10; G01N 2333/52

(57) ABSTRACT

Pharmaceutical compositions and methods for regulating somatic cell reprogramming in mammals, and in particular, for positively and negatively regulating cell reprogramming in human cells in vivo and in vitro. The invention also provides PHF20-derived compositions and methods useful for cancer immunotherapies, including breast cancer therapies in particular.

### PRIORITY INFORMATION

This application claims priority of pending U.S. App. Ser. No. 61/769,545, filed Feb. 26, 2013, the contents of which are incorporated herein by reference.

### DESCRIPTION OF RELATED ART

Both human and mouse somatic cells can be reprogrammed to a pluripotent embryonic stem cell (ESC)-like state, giving rise to induced pluripotent stem cells (iPSCs), by the use of four key transcription factors: Oct4, Sox2, Klf4 and c-Myc (Okita et al., 2007; Takahashi et al., 2007b; Takahashi and Yamanaka, 2006: Yu et al., 2007). Because of their similarity to ESCs in terms of gene expression profile, epigenetics/genetic marks, and their ability to self-renew and differentiate into many different cell types, iPSCs hold great promise for human disease modeling, drug screening and perhaps therapeutic applications (Plath and Lowry, 2011; Robinton and Daley, 2012). Although somatic cell reprogramming can be achieved by several strategies, including inducible expression of four transcription factors, protein transduction and microRNA (miRNA) expression, with or without small-molecule compounds (Robinton and Daley, 2012; Stadtfeld and Hochedlinger, 2010), its efficiency, and the kinetics of iPSC generation are still suboptimal. This suggests the existence of substantial genetic and epigenetic barriers during reprogramming (Hanna et al., 2009; Smith et al., 2010).

Many factors, including cell proliferation and cycling, mesenchymal-to-epithelial transitions and epigenetic regulation of histone modification and DNA methylation, influence reprogramming efficiency (Papp and Plath, 2011; Stadtfeld and Hochedlinger, 2010). Transiently enforced expression of reprogramming factors leads to separable events, beginning with mesenchymal-to-epithelial transitions associated with loss of the somatic marker THY I, followed by the activation of embryonic markers such as alkaline phosphatase (AP) and stage-specific embryonic antigen 1 (SSEA1) (Li et al., 2010; Plath and Lowry. 2011). Induction and maintenance of endogenous pluripotency genes such as Nanog and Oct4 require further epigenetic reprogramming changes at the DNA methylation and histone modification levels (Stadtfeld and Hochedlinger, 2010). Failure to achieve these epigenetic changes in a timely manner can lead to partially reprogrammed iPSCs.

Global analysis of euchromatin dynamics during the reprogramming process has revealed orchestrated epigenetic changes at the histone modification level (Gaspar-Maia et al., 2011; Hemberger et al., 2009: Hochedlinger and Plath, 2009; Koche et al., 2011). Ectopic expression of the chromatin remodeling proteins Brg-1 and Baf155, for example, enhances the efficiency of four factor-mediated reprogramming (Gaspar-Maia et al., 2009; Singhal et al., 2010). Both ESCs and iPSCs contain “bivalent domains,” where nucleosomes are marked with trimethylation at histone3-lysine27 (H3K27me3) and histone3-lysine4 (H3K4me3) (Gaspar-Mafia et al., 2011; Hochedlinger and Plath, 2009). While the Polycomb group (PcG) complex mediates H3K27 methylation and inhibits gene repression (Cao et al., 2002; Margueron and Reinberg, 2011), Jmjd3 and Utx mediate H3K27 demethylation (Agger et al., 2007; Hong et al., 2007; Jepsen et al., 2007; Kouzarides, 2007; Lan et al., 2007). Thus, given the importance of epigenetic factors in defining cell lineages, it is reasonable to suggest that some of these factors are required for efficient somatic reprogramming, while others may function as negative regulators. Removal of such roadblocks to successful reprogramming will require increased insight into the molecular mechanisms by which epigenetic factors control cell lineage and hence the dynamic process of reprogramming.

### SUMMARY OF THE INVENTION

Jmjd3 was identified as a potent negative regulator of reprogramming. Jmjd3-deficient mouse embryonic fibroblasts (MEFs) produced significantly more iPSC colonies than did wild-type cells, while ectopic expression of Jmjd3 markedly inhibited reprogramming. The inhibitory effects of Jmjd3 are produced through both histone demethylase-dependent and -independent pathways, the latter of which is entirely novel and involves Jmjd3 targeting of PHF20 for ubiquitination and degradation via recruitment of an E3 ligase, Trim26. PHF20-deficient MEFs could not be converted to fully reprogrammed iPSCs, even with knockdown of Jnmjd3, Ink4a or p21, indicating that this protein exerts dominant effects on reprogramming. The present invention accordingly provides a method for inducing pluripotent stem cell formation, comprising inhibiting or preventing the expression of expression of Jmjd3 gene in a cell, or inhibiting the activity of the JMJD3 protein in a cell, e.g. by adding a JMJD3 antagonist to a cell culture.

PHF20 was further found to be overexpressed in more than 90% of breast cancer tissue and acts as a new breast cancer antigen with an important role in the mediation of a strong anti-tumor immune response. Immuno-therapies of breast cancer targeting PHF20 is thus provided. In one embodiment, a combination immunotherapy that will generate a PHF20 antigen-specific immune response while inhibiting breast cancer tumor growth is provided. In another embodiment, dendritic cells (DC) loaded with nanoliposomes containing the PHF20 peptide and siRNAs with anti-PD-1 (programmed death-1) blockage, or anti-human PD1 (anti-PD1) antibody, are administered to a patient in need thereof, where the PHF20/DC vaccination will enhance the precursors of antigen-specific T cells, while anti-PD-1 blockade will increase antigen-specific T cell response, inhibiting tumor growth and reducing non-specific immune response or side effects.

In another embodiment, the cancer-specific PHF20 antigen may be targeted by a suitable preparation comprising an antibody against PHF20, preferably a humanized, or a human, monoclonal antibody.

The present invention also provides a pharmaceutical composition comprising a PHF20 peptide which is derived from the PHF20 protein and is a cytotoxic T lymphocyte (CTL) epitope, a pharmaceutical acceptable excipient. The PHF20 peptide, preferably a human peptide, is able to stimulate T cells so that the T cells are able to recognize T2 cells loaded with a PHF20 peptide, or PHF20-positive breast cancer cells.

In one embodiment, the present invention provides a vaccine or a pharmaceutical composition comprising a PHF20 peptide, a nucleic acid molecule encoding the PHF20 peptide, or an expression vector comprising a nucleic acid encoding the PHF20 peptide.

In one embodiment, the present invention provides an isolated T-cell, preferably a CTL, specific for a PHF20 peptide, or an isolated T-cell produced by stimulating peripheral blood mononuclear cells (PBMCs) with a PHF20 peptide.

The present invention also provides a method of treating breast cancer, comprising (a) isolating a cell population containing or capable of producing CTLs and/or T_{H} cells from a subject; (b) treating the cell population with a PHF20 peptide, optionally together with a proliferative agent; (c) screening the cell population for CTLs or T_{H} cells or their combination, with specificity to a PHD20 peptide; and (d) administering the cell population to a patient suffering from cancer.

Alternatively, the above method may comprise: (a) isolating a cell population containing or capable of producing CTLs, T_{H} cells or their combination from a subject; (b) treating the cell population with PHF20 peptide, optionally together with a proliferative agent; (c) screening the cell population for CTLs, T_{H} cells or their combination with specificity to PHF20 peptide; (d) cloning the T cell receptor (TCR) genes from the screened CTLs, T_{H} cells or their combination with specificity to the PHF20 peptide described herein; (e) transducing the TCR gene cloned in step (c) into either: i. cells from the patient; or ii. cells from a donor; or iii. eukaryotic or prokaryotic cells for the generated mTCRs from step (e) to a patient suffering from breast cancer.

### BRIEF DESCRIPTION OF THE DRAWINGS

FIG. 1 shows the identification of Jmjd3 and other key epigenetic factors that regulate reprogramming. Ectopic expression of Jmjd3 inhibits reprogramming. The data in FIG. 1H and FIG. 1I are reported as the means+SD of three independent experiments. Asterisks indicate significant differences from the control (*p<0.05, **p<0.01 ***p<0.001 by Student's t test).

FIG. 2 shows Jmjd3 ablation enhances the efficiency and kinetics of reprogramming. The data in FIG. 2B and FIG. 2C are reported as means+SD of three independent experiments. Asterisks indicate significant differences between groups (*p<0.05, **p<0.01 by Student's t test);

FIG. 3 shows the identification of Jmjd3 targets responsible for enhanced reprogramming. The data in FIG. 3B, FIG. 3D, FIG. 3E, and FIG. 3F are reported as means±SD of three independent experiments. Asterisks indicate significant differences between groups (*p<0.05, **p<0.01, ***p<0.001 by Student's t test).

FIG. 4 shows PHF20 is essential for maintenance and reprogramming of iPSCs. The data in FIG. 4D, FIG. 4F, FIG. 4H, FIG. 41, FIG. 4J, FIG. 4K, and FIG. 4L are plotted as means±SD of three independent experiments. Asterisks indicate significant differences between groups (*p<0.05, **p<0.01 by Student's t test).

FIG. 5 shows Jmjd3 interacts with PHF20 and causes its degradation.

FIG. 6 shows Jmjd3 targets PHF20 for ubiquitination by recruiting an E3 ligase, Trim26.

FIG. 7 shows PHF20 is required for Oct4 expression during reprogramming by interacting with Wdr5. The data in FIG. 7A, FIG. 7C-FIG. 7E, FIG. 7I and FIG. 7J are plotted as means±SD of three independent experiments. Asterisks indicate significant differences between groups (*p<0.05, **p<0.01, by Student's t test).

FIG. 8 shows that PHF20 is highly expressed in breast cancer cells. FIG. 8A shows PHF20 mRNA expression in breast cancer cells as determined by real-time PCR. FIG. 8B is a Western blot analysis of PHF20 in breast cancer cell lines and normal cells. MCF-10A is a normal breast cell line.

FIG. 9 shows the generation and characterization of PHF20-specific T cells. (FIG. 9A) PHF20₉₃₈₋₉₄₆-specific CD8⁺ T cells were generated from normal donor PBMCs after in vitro stimulation with PHF20 peptides, and used to determine the recognition of T2 cells loaded with different concentrations of PHF20₉₃₈₋₉₄₆, peptide, or a control peptide as a negative control. (FIG. 9B) PHF20₉₃₈₋₉₄₆-specific T cells were cultured alone in medium or co-incubated with HLA-A2⁺ PHF20⁺ (MCF-7) or HLA-A2⁻ PHF20⁺ (DU4475, MDA-MB-361) breast cancer cell lines. A normal breast epithelial cell line MCF-10A is included as a control. T cell activity was determined by measuring cytokine (IFN-γ) release. (FIG. 9C) PHF20₉₃₈₋₉₄₆-specific CD8⁺ T cells were tested for cytotoxicity against MCF-7 by the LDH assay. HLA-A2 negative PHF20⁺ PC3 cells were used as a negative control in the LDH assay. Data are plotted as means t SD. Results are representative of three independent experiments. *P<0.05, **P<0.01, ***P<0.001 versus controls.

FIG. 10 shows enhancing antigen-specific immune response by knocking down negative regulators in DCs. Such an antitumor immunity may be further improved by nanotechnology-based delivery system.

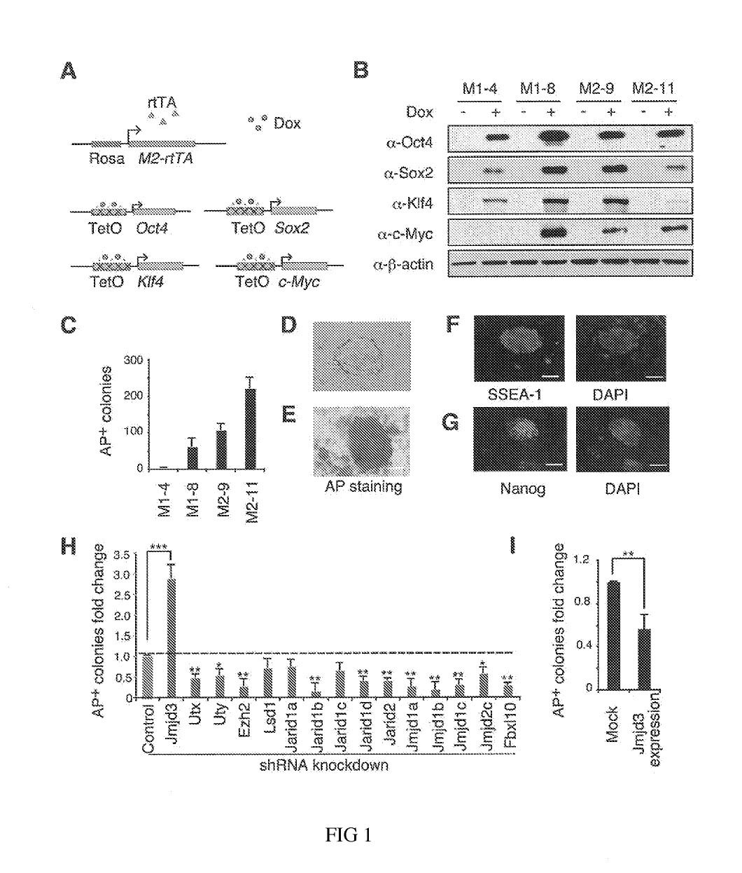

FIG. 1I illustrates a MSV delivery system and its use for generating potent antitumor immunity in a therapeutic tumor model. (A) A schematic presentation of MSV with or without liposomes. (B) Antitumor immunity generated by DCs loaded with MSV/liposomes containing TRP-2, CpG (a ligand of TLR9) and MPLA (monophosphoryl lipid A, a ligand of TLR4). Mice were intravenously injected with B16 tumor cells (0.3×10⁶ in 200 μl PBS per mouse). 4 days later, these tumor-bearing mice were immunized with DCs (0.3×10⁶) loaded with the indicated peptides or nanoparticles. 14 days after vaccination, lung metastases were examined for lung metastasis. (C) Number of B16 lung mets among different treatment groups. The number of 250 represents “too many to count” in 4 groups.

FIG. 12 illustrates combination therapy of DC/PHF20 vaccination with anti-PD-1 blockade.

FIG. 13 shows two schedules for DC/peptide vaccination and anti-PD-1 combination.

FIG. 14 Generation of PHF20-specific T cell response in HLA-A2 Tg mice. HLA-A2 Tg mice were immunized with DC/PHF20 peptide. Eight days later, T cells were isolated from splenocytes and tested for their ability to recognize PHF20 or irrelevant peptide. T cells were stained with anti-CD8-FITC and then intracellular stained with anti-IFN-γ-PE. FACS analysis was performed after gating on CD8+ T cell population.

### DESCRIPTION OF THE INVENTION

### Jmjd3 Negatively Regulates Somatic Cell Reprogramming

Using a shRNA knockdown screen in Tet-O-4F MEFs, a number of histone-modifying proteins were identified that are required for the reprogramming of somatic cells to iPSCs. Only one, however, Jmjd3, functioned as a negative regulator of this process.

Jmjd3 μlays a critical role in the upregulation of Ink4a/Arf by modulating the levels of H3K27 trimethylation in the promoter (Agger et al., 2009; Barradas et al., 2009). It interacts with other target genes by demethylating H3K27 trimethylation and promoting transcriptional elongation through interaction with KIAA1718 (Chen et al., 2012). These effects on the expression of Ink4a/Arf and p21, in turn, induce senescence and inhibit reprogramming (Hong et al., 2009; Kawamura et al., 2009; Li et al., 2009; Marion et al., 2009; Utikal et al., 2009), consistent with the demonstration that Jmjd3 ablation reduces cell senescence and promotes reprogramming of Jmjd3-deficient MEFs through downregulation of Ink4a and p21 expression. However, several lines of evidence are provided indicating that Jmjd3 can regulate reprogramming through a previously unrecognized, histone demethylase activity-independent pathway. First, the combined knockdown of Jmjd3 with Ink4a or p21 resulted in significantly more iPSC colonies than did knockdown of any single gene alone, indicating that the reprogramming function of Jmjd3 exceeds that predicted from its upregulation of Ink4a or p21. Second, although ectopic expression of full-length Jmjd3 in Jmjd3-deficient MEFs restored Ink4a/Arf expression and strongly inhibited the efficiency of reprogramming, the Jmjd3 mutants Jmjd3-AlmjC and Jmjd3-111390A, defined by their lack of H3K27me3 demethylase activity and inability to upregulate hk4a/Arf expression, could still inhibit reprogramming in Jmjd3-deficient MEFs. Jmjd3 exploits both demethylase activity-dependent and -independent mechanisms to regulate somatic cell reprogramming, with the latter having the dominant role. An extensive search for target molecules that might be involved in a Jmjd3-mediated but demethylase activity-independent pathway led to the identification of PHF20. Knockdown of PHF20 expression induced rapid differentiation of ESCs and iPSCs, while treatment of ESCs and iPSCs with RA or removal of LIP led to downregulation of PHF20. Oct4 and Nanog expression, suggesting a role for PHF20 expression in the maintenance of the pluripotent state. PHF20 was first identified as an antibody-reactive protein that is highly expressed in several types of cancer including I oblastoma hepatocellular carcinoma, and medulloblastoma (Fischer et al., 2001; Wang et al., 2002). PHF20 has since been identified as a histone code reader that specifically recognizes the dimethylation of H3K4. H3K9, H4K20, and H4K79 (Adams-Cioaba et al., 2012; Kim et al., 2006). Recent studies show that it also recognizes dimethylated p53 at K370 and K382, and regulates p53 protein at both the transcriptional and posttranscriptional levels in response to DNA damage (Li et al., 2012b; Park et al., 2012). Mice with PHF20 ablation die shortly after birth (Badeaux et al., 2012). Furthermore, ESC lines could not be generated from PHF20 knockout mice. Consistent with this, it was also shown that PHF20 deficiency almost completely inhibited reprogramming of PHF20-deficient MEFs, which suggested an absolute requirement for this factor in iPSC reprogramming and generation of ESCs.

Jmjd3 has been shown to play an important role in T cell-lineage commitment by interacting with T-bet, a master regulator of CD4⁺ T helper 1 (Th1) cells, as well as Brg-1, a key catalytic subunit of the chromatin-remodeling BAP complex, in a demethylase activity-independent manner (Miller et al., 2010). It this example, Jmjd3 is shown to interact with PHF20, targeting it for ubiquitination and proteasomal degradation. Although both the N- and C-terminal regions of Jmjd3 protein can bind to the N-terminus of PHF20, Jmjd3 itself cannot ubiquitinate PHF20 in a K48-linkage. Instead, it recruits an E3 ligase, Trim26, to PHF20 for K48-linked polyubiquitination. Indeed, knockdown of Trim26 abolishes PHF20 ubiquitination and degradation, thus enhancing iPSC reprogramming. Further studies demonstrated that like full-length Jmjd3, certain Jmjd3 mutants (Jmjd3-AJmjC and Jmjd3-H1390A), but not Jmjd3-N, Jmjd3-M, or Jmjd3-C, target PHF20 for ubiquitination. These results emphasize the importance of Jmjd3-Trim26 mediated ubiquitination in the negative regulation of reprogramming.

Fully reprogrammed iPSCs are accompanied by changes in distinct DNA methylation patterns associated with reactivation of endogenous Oct4 and several other ESC marker genes (Plath and Lowry, 2011; Stadtfeld and Hochedlinger, 2010). How, then, does PHF20 deficiency lead to failure to reactivate these endogenous marker genes, thus imposing a major barrier to successful reprogramming? A recent study shows that exogenous Oct4 together with other key reprogramming factors first induce Wdr5 expression in MEFs, which in turn forms a Wdr5-Oct4 complex that binds to the Oct4 promoter, leading to reactivation of endogenous Oct4 expression (Ang et al., 2011). To directly link PHF20 to Oct4 expression, it was shown that PHF20 not only binds to the Oct4 promoter region, but also specifically interacts with Wdr5 and MOF. A recent study shows that MOF is required for ESC self-renewal and function, and regulates Nanog expression (Li et al., 2012a). Deletion of PHF20 reduces the ability of Wdr5 and MOF to bind to the Oct4 promoter, suggesting a critical requirement for this protein in reactivation of endogenous Oct4 expression and hence for successful generation of fully reprogrammed iPSCs. Consistent with this notion, the results presented in this example further demonstrated that PHF20 deficiency resulted in failure to reactivate expression of many endogenous ESC maker genes during reprogramming, although the expression of endogenous Sox2 and Nanog can be rescued by expression of exogenous Oct4 (in the presence of Dox). This suggested that PHF20 affects expression of many key ESC genes directly or indirectly. ChIP-PCR and ChIP-seq analyses showed that PHF20 and Wdr5 bind to the Oct4 promoter, but not to the promoter regions of Sox2, Nanog, Dnmt3i, Esgl, Eras, Rexl or Crinto. In addition, ChIP-seq analysis revealed that both PHF20 and Wdr5 bind to several key epigenetic factor genes, including Baf155, Brg-1 and Sal4. Hence, these findings explain why the incompletely reprogrammed phenotype of PHF20-deficient MEFs cannot be rescued by overexpression of Oct4 or 4F (OSKM), and further suggest that PHF20 functions as an upstream regulator that controls Oct4 and many other critical ESC marker genes, thus providing a mechanistic link between Jmjd3-mediated PHF20 degradation and deficient reprogramming.

Based on these findings, a working model has been proposed to explain how the Jmjd3-PHF20 axis regulates iPSC reprogramming. Increased expression of Jmjd3 due to 4F-mediated reprogramming in WT MEFs initiates at least two distinct pathways (FIG. 7K):

1) Jmjd3 upregulates Ink4a/Arf and p21 by modulating H3K4 and H3K27 methylation through its H3K27me2/3 demethylase activity. Increased amounts of Ink4a, Arf and p21 induce cell senescence or apoptosis and reduce cell proliferation, thus decreasing the efficiency and kinetics of reprogramming;

2) Jmjd3 protein also targets PHF20 for ubiquitination and degradation by recruiting an E3 ligase, Trim26, in a 113K27 demethylase activity-independent manner. The resultant decrease in PHF20 protein leads to the loss or negligible expression of endogenous Oct4, thereby greatly reducing reprogramming efficiency. It was concluded that the demethylase activity-dependent and -independent pathways used by Jmjd3 act in concert to potently restrain the kinetics and efficiency of reprogramming. The observations that Jmjd3 loss reduces cell senescence and apoptosis and increases cell proliferation, and that increased amounts of PHF20 lead to reactivation of endogenous Oct4 expression, provide opportunities to enhance the outcome of somatic cell reprogramming overall.

### PHF20 as Immunotherapeutic Targets for Cancer Treatment

The inventors discovered that PHF20 (plant homeodomain finger-containing protein 20) is over-expressed in more than 90% of breast cancer tissue and acts as a new breast cancer antigen with an important role in the mediation of a strong anti-tumor immune response. Immuno-therapies of breast cancer targeting PHF20 is thus provided. In one embodiment, a combination immunotherapy that will generate a PHF20 antigen-specific immune response while inhibiting breast cancer tumor growth is provided. In another embodiment, dendritic cells (DC) loaded with nanoliposomes containing the PHF20 peptide and siRNAs with anti-PD-1 (programmed death-1) blockage, or ani-human PD1 (anti-PD1) antibody, are administered to a patient in need thereof, where the PHF20/DC vaccination will enhance the precursors of antigen-specific T cells, while anti-PD-1 blockade will increase antigen-specific T cell response, inhibiting tumor growth and reducing non-specific immune response or side effects. In another embodiment, the cancer-specific PHF20 antigen may be targeted by a suitable preparation comprising an antibody against PHF20, preferably a humanized, or a human, monoclonal antibody.

The present invention further provides a pharmaceutical composition, comprising a vaccine for the treatment or management of breast cancers. The vaccine of the present invention comprises, among other components, a PHF20 peptide, which can be any peptode that is derived from the PHF20 protein (described in more details below), and serves as a cytotoxic T lymphocyte (CTL) epitope. A PHF20 peptide of the present invention can be used to in vitro stimulate T cells, which will then be able to recognize T2 cells loaded with different concentrations of a PHF20 peptide, or PHF20-positive breast cancer cells.

The software NetMHC version 3.2 (cbs.dtu.dk/services/NetMHC-3.2) was used to predict and select 9mer peptides binding to allele HLA-A0201 using Artificial Neural Networks. Any peptide with a threshold of 50 nM or lower is designated as a strong binder, and those with threshold score of 500 nM were not selected. Ten (10) 9mer peptides derived from protein PHF20 are selected, and their sequences are shown in Table 1 below.

TABLE 1
Amino Acid Sequence of PHF20 Peptides

| | | | Affinity | Allele |
|---|---|---|---|---|
| Peptide# | Position | Peptide sequence | (nM) | restriction |
| PHFP 1 | 863-871 | ALDDAVNPL (SEQ ID NO: 1) | 12 | HLA-A*0201 |
| PHFP 2 | 938-946 | WQFNLLTHV (SEQ ID NO: 2) | 17 | HLA-A*0201 |
| PHFP 3 | 277-285 | TLQPITLEL (SEQ ID NO: 3) | 29 | HLA-A*0201 |
| PHFP 4 | 751-759 | RVIEVLHGL (SEQ ID NO: 4) | 31 | HLA-A*0201 |
| PHFP 5 | 744-752 | QLLGDVQRV (SEQ ID NO: 5) | 49 | HLA-A*0201 |
| PHFP 6 | 941-749 | NLLTHVESL (SEQ ID NO: 6) | 52 | HLA-A*0201 |
| PHFP 7 | 87-95 | FQINEQVLA (SEQ ID NO: 7) | 101 | HLA-A*0201 |
| PHFP 8 | 934-942 | SQHQWQFNL (SEQ ID NO: 8) | 142 | HLA-A*0201 |
| PHFP 9 | 949-957 | LQDEVTHRM (SEQ ID NO: 9) | 402 | HLA-A*0201 |
| PHFP 10 | 116-124 | YTVKFYDGV (SEQ ID NO: 10) | 409 | HLA-A*0201 |

In a specific embodiment the PHF20 protein from which a PHF20 peptide is derived is the human PHF20 protein. In another embodiment, the PHF20 peptide comprises the amino acid WQFNLLTHV (SEQ ID NO:2).

The vaccine of the present invention can, in some embodiment, be used in a combination therapy.

In some embodiments, a composition provided herein comprises PHF20 peptide-based breast cancer vaccine. In other embodiments, a composition provided herein comprises the vaccine and a helper T cell epitope, an adjuvant, and/or an immune response modifier. In other embodiments, a composition provided herein comprises an immune response modifier. The pharmaceutical compositions provided herein are suitable for veterinary as well as human administration.

In one aspect, the PHF20 peptide-based vaccine described herein is used in preventing, treating, and/or managing breast cancer in a subject in need thereof, the method comprising administering to the patient a prophylactically effective regimen or a therapeutically effective regimen. The patient may be diagnosed with breast cancer, or simply be at risk of developing breast cancer. The patient may have undergone another therapy which may have been effective or ineffective.

The immune system plays a critical role in the recognition, control and destruction of cancer cells. Harnessing the immune system to eradicate malignant cells is becoming a powerful approach to cancer therapy, but until recently, it had met with only sporadic clinical success (Rosenberg, 2011: Lesterhuis et al., 2011; Di Lorenzo et al., 2011). Recent FDA approval of the immunotherapy-based drug sipuleucel-T (Provenge) and ipilimumab (Yervoy, anti-CTLA-4) for the treatment of metastatic prostate cancer and melanoma, respectively, represents milestones in the field of cancer immunotherapy (Kantoff et al., 2010; Hodi et al., 2010). However, immunotherapy of breast cancer is still lacking.

Although a number of breast tumor antigens have been identified as immunotherapy targets, they are expressed in only a small fraction of breast cancer. For example, HER-2/neu is expressed only in 20% of breast cancers. Similarly, NY-ESO-1 and MAGE-A family antigens are expressed in only a small fraction (2-6%) of breast cancer (Chen et al., 2011), limiting their use in more than 80% breast cancer patients. Thus, one of the major hurdles in the field of breast cancer immunotherapy is to identify novel breast cancer antigens that can be applied to the majority of cancer patients. To address this key issue, the inventors recently identified PHF20 (plant homeodomain finger-containing protein 20) as an immunogenic breast cancer antigen that is overexpressed in more than 90% of breast cancer. Both MHC class I- and II-restricted T cell epitopes have been identified. PHF20-specific T cells are capable of recognizing PHF20-positive breast cancer cells. PHF20 is also known as glioma-expressed antigen 2 (GLEA2) and hepatocellular carcinoma antigen 58 (HCA58), and can elicit strong antibody response in cancer patients (Fischer et al., 2001; Wang et al., 2002). Importantly, autoantibody against PHF20 response significantly correlated with prolonged survival in patients with glioblastoma (Pallasch et al., 2005). However, among many factors that may contribute to the relatively low clinical efficacy of peptide-based vaccines and even FDA-approved vaccine (such as sipuleucel-T) (Buonerba et al., 2011) potent multiple inhibitory mechanisms, including T-cell co-inhibitory molecules CTLA-4 and PD-1 (programmed death 1) signaling and regulatory T (Treg) cells, are a major obstacle to improving therapeutic efficacy of cancer vaccines and drugs (Curiel et al., 2004; Wang et al., 2004; Zou, 2006; Wang and Wang, 2007). PD-1 is a key immune checkpoint molecule expressed by activated T cells (Dong et al., 1999), and mediates its immune suppression by interacting with its ligand PD-L1 (B7-h1) expressed on tumor cells and stromal cells (Dong et al., 2002). Recent clinical trials with anti-PD-1 antibody show durable tumor regression with objective clinical response rate of 18-28% for lung cancer, melanoma and renal-cell cancer (Topalian et al., 2012; Brahmer et al., 2012).

Hence, the key to improving the clinical efficacy of PHF20-based immunotherapy is 1) to modulate the capacity of DCs to generate robust immune response; and 2) to further amplify antigen-specific immunity through blockade of PD-1-mediated inhibitory signaling. PHF20 vaccines generate breast cancer-specific immunity, which can be further enhanced by knockdown of negative signaling in DCs and by blockade of co-inhibitory signaling in T cells, thus generating potent and lasting antigen-specific immune responses against breast cancer.

A phase I clinical trial for metastatic prostate cancer patients using NY-ESO-1 peptide vaccines has recently been conducted. Such peptide vaccines are safe and capable of eliciting tumor-specific immunity, but like other protein/peptide-based vaccine studies, the clinical response rate remains to be improved. Because NY-ESO-1 is expressed in only a small fraction (2-6%) of breast cancer, it is not suitable for immunotherapy of breast cancer. To this end, PHF20 has been identified as an immunogenic target, which is expressed in more than 90% of breast cancer samples or cell lines. Therefore, PHF20 protein/peptides-based vaccines are suitable for inducing tumor-specific T cell response, which can be further enhanced by knockdown of negative signaling in DCs and by anti-PD-1 blockade. To promote the translational research, GMP-grade PHF20 peptides have been prepared, and GMP grade anti-PD-1 and anti-PD-L1 antibodies are available for use.

Although immune suppression and negative immune regulation are fundamentally important to maintaining a homeostatic balance between host immunity and tolerance, they also pose major obstacles to the development of potent vaccines and drugs (Sakaguchi et al., 2004). This is particularly true for cancer because PD-1 signaling effectively dampens the immune response induced by cancer peptide/protein vaccines (Zou, 2006; Wang and Wang, 2007). The studies presented here are the first to target PHF20 as a novel immunogenic antigen for immunotherapy of breast cancer, and show that PHF20-specific antitumor immunity can be further enhanced by blocking negative signaling in DCs and T cells, thus releasing the brake on the generation and proliferation of antigen-specific T cell response. Most importantly, combined therapy of DC/peptide vaccines with blockade of PD-1 negative signaling could produce optimal antigen-specific antitumor immunity. Such a combination therapy represents an entirely novel approach to the treatment of metastatic breast cancer.

These studies also directly address one of the major problems—immune checkpoint and immune suppression—in the field of cancer immunotherapy. This invention opens new opportunities for the treatment of metastatic breast cancer through blocking immune suppression and negative regulators, leading to the development of novel antigen-specific therapeutic anti-cancer vaccines/drugs.

PHF20 as a New Breast Cancer Antigen:

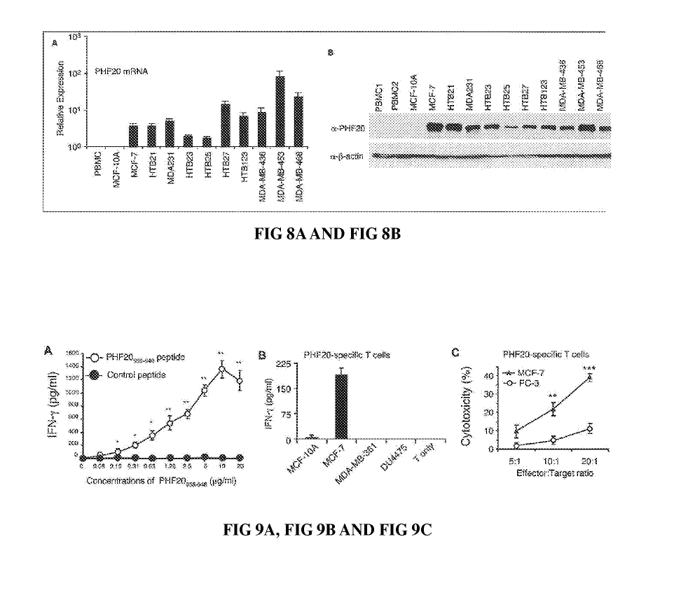

Because HER-2/neu is expressed only in 20% of breast cancers, and NY-ESO-1 and MAGE-A family antigens are expressed in only a small fraction (2-6%) of breast cancer, it is fundamentally important to identify breast cancer antigens that can be applied to the majority of breast cancer patients for immunotherapy. To this end, PHF20 was identified as a new breast cancer antigen. PHF20 is highly expressed in breast cancer cells, but shows little or no expression in normal breast cell line (MCF-10A), PBMCs or normal tissues with the exception of testis. High PHF20 expression at the mRNA and protein levels in breast cancer cells was demonstrated by real-time PCR and Western blot analysis, as shown in FIG. 8A and FIG. 8B.

PHF20-Specific T Cells Recognize Breast Cancer:

To determine the immunogenicity of PHF20, T cells from the PBMCs of HLA-A2⁺ healthy donors were generated and PHF20-specific T cells were shown to be capable of recognizing T cells pulsed with PHF20₉₃₈₋₉₄₆ peptide (WQFNLLTHV), and PHF20-positive breast cancer cells (FIG. 9A and FIG. 9B). Furthermore, PHF20-specific T cells could lyse HLA-A2⁺ and PHF20⁺ MCF-7 breast cancer cells (FIG. 9C).

PHF20 is Required for Stem Cell Reprogramming:

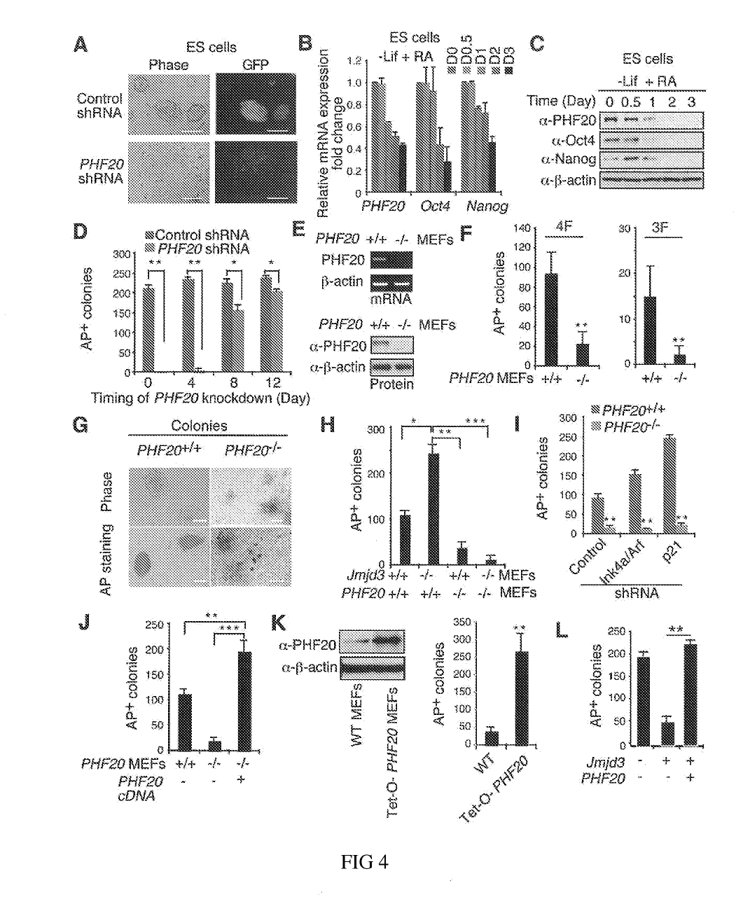

It was shown that PHF20 is required for the maintenance and renewal of embryonic stem cells (ESC) or inducible pluripotent stem cells (iPSCs). Deletion of PHF20 resulted in differentiation of iPSCs/ESCs and down regulation of several stem cell markers (FIG. 4G and FIG. 4C). Loss of PHF20 in MEFs blocked cellular reprogramming to generate iPSCs, which could be rescued by overexpression of PHF20 cDNA (FIG. 4J). These results suggest that PHF20 is required for the generation and maintenance of ESCs and iPSCs, raising the possibility that PHF20 may play an important role in cancer stem cell renewal and maintenance.

Inhibitory Molecules or Negative Regulators:

Co-inhibitory molecules of T cell activation such as CTLA-4 and PD-1 (programmed cell death 1) have been targeted for antibody drugs that block negative signaling in T cells. Similarly, knockdown of negative regulators of innate immune signaling such as A20 in dendritic cells (DCs) increases their capacity to resist immune suppression and induce robust antitumor immunity (Song et al., 2008). Several important negative regulators were recently identified, including NLRC5, NLRX1 and NLRP4, all of which are members of the NOD-like receptor protein family. NLRC5 and NLRX1 potently inhibit NF-κB activation by interaction with IκB kinase (IKK) complex through distinct mechanisms (Cui et al., 2011; Xia et al., 2011). Importantly, they also inhibit type I interferon signaling by targeting different receptors or adaptor molecules. NLRC5 inhibits type I IFN signaling by directly interacting with RIG-I and MDA-5 for their function (Cui et al., 2011), while NLRX1 interacts with MAVS, a key adaptor molecule, and inhibits type I IFN signaling (Moore et al., 2008). Knockdown or knockout of these negative regulators enhances both NF-κB and type I IFN signaling and produces more proinflammatory cytokines (Cui et al., 2012; Tong et al., 2012).

Phase I Clinical Trial of NY-ESO-1 Peptides in Prostate Cancer Patients:

Phase I trials using NY-ESO-1 recombinant protein or synthetic peptides have been conducted in melanoma and ovarian cancer (Davis et al., 2004; Khong et al., 2004; Odunsi et al., 2007; Valmori et al., 2007). A phase I clinical trial was recently completed for metastatic prostate cancer patients that was designed to evaluate the safety and feasibility of combined use of MHC class I and/or class II NY-ESO-1 peptides. Nine patients were enrolled in this study. It was found that peptide vaccines were well tolerated. The median PSA doubling times (PSA-DT) was prolonged compared to the baseline in 6 patients, including a decrease in PSA level in 2 patients. Strong NY-ESO-1-specific T cell responses were observed in 6 of the 9 patients. These encouraging clinical results have provided impetus for testing whether PHF20 peptide vaccines can elicit strong immune response in breast cancer, because the low expression frequency of NY-ESO-1 in breast cancer prevents it from being used as an immune target for breast cancer.

Since PHF20 peptides can sensitize T cells from healthy donor PBMCs, the precursors of HLA-A2-restricted PHF20-specific T cells in cancer patients were higher than in the PBMCs of healthy donors. If so, such antigen-specific T cells could be readily detected, induced and expanded following in vitro stimulation with PHF20 peptides. In the case of NY-ESO-1, it was found that human NY-ESO-1-specific T cells could be readily detected from PBMCs derived from patients who developed antigen-specific antibody (Zeng et al., 2000).

PHF20 Peptides Readily Induced Antigen-Specific T Cells in the PBMCs of Cancer Patients:

Although the majority of self-antigen-specific T cells are deleted through a central tolerance mechanism, some self-antigen-specific T cells escape from such a tolerance mechanism, and are circulated in the peripheral blood. These T cells in general exhibit relatively low T-cell receptor (TCR) affinity. When cancer-associated self-antigens such as PHF20 are overexpressed in cancer cells, they might induce and increase the affinity and precursor of such antigen-specific T cells in the PBMCs of cancer patients, compared with healthy donors.

20 PBMCs (10 HLA-A2⁺ healthy donors and 10 HLA-A2⁺ breast cancer patients) are collected, and approximately 2.5×10⁵ PBMCs of each sample are plated in a 96-well flat-bottomed plate in the presence of 10 μg/mL peptide. On days 7 and 14, 1×10⁵ irradiated PBMCs are pulsed with 10 μg/mL peptide, washed twice, and added to each well. IL-2 at 120 IU/mL is added on day 8, day 11, day 15, and day 18. At each cycle of stimulation, cells are harvested and incubated with target cells overnight before the supernatants are taken for cytokine release assays. Both HLA-A2-matched and mismatched tumor cell lines or EBV transformed B cells are co-cultured with T cells overnight. The cell supernatants are harvested for cytokine release assays (GM-CSF, IFN-γ, IL-4, 11-17 and IL10) using ELISA kits. Antigen-specific T cells are further determined by ELISPOT, intracellular cytokine staining, and tetramer staining. The ELISPOT and intracellular staining assay are reliable methods for assessing functional antigen-specific T cells, while tetramer staining will determine the percentage of antigen-specific T cells in total population without the knowledge of T cell function. These experiments demonstrate that PHF20 peptides induce and expand antigen-specific T cells present in the PBMCs of breast cancer patients more readily than in the PBMCs of healthy donors.

To test whether the TCR affinity of PHF20-specific T cells derived from the PBMCs of breast cancer patients are higher than that of healthy donor PBMCs, peptides are diluted at different concentrations (for example, 10, 3, 1, 0.3, 0.1, 0.03, 0.01, 0.003, 0.001 μM in serum-free medium), and pulsed onto HLA-A2-matched T2 or EBV-B cells for 90 min, and washed three times. T cells derived from healthy donors and breast cancer patients are added to the wells with different concentrations of peptides. After co-culture with T cells overnight, cytokine release in supernatants may be measured by ELISA kit.

PHF20-Specific Antibody Response is Associated with High Precursors of Antigen-Specific T Cell Response in Breast Cancer Patients.

To determine PHF20-specific antibody response in healthy donors and breast cancer patients, 80 sera from cancer patients and 20 sera from healthy donors (as a control), are collected in collaboration with Dr. Jenny Chang at TMHRI. The detailed experimental procedure is similar to that previously described for measuring NY-ESO-1 antibody response³⁰. A positive reaction is defined as an O.D. value against PHF20 that exceeds the mean O.D. value plus three times SDs of normal donors at serum dilutions of both 1/25 and 1/250. Once breast cancer patients with a high titer of anti-PHF20 antibody are identified, patients with anti-PHF20 antibody in the sera are tested to determine if they contain high precursor frequency of PHF20-specific T cells, compared with cancer patients without anti-PHF20 antibody. Breast cancer patients with anti-PHF20 antibody are also tested to show better survival. Overall, these studies show immunotherapy of breast cancer using PHF20 protein/peptides is effective, and anti-PHF20 antibody and antigen-specific T cell responses are also important in the control of breast cancer.

HLA-A2 transgenic (Tg) mice have been successfully used as a preclinical model for cancer vaccine studies. More recently, humanized NGS-A2 mice available at the Jackson Laboratory have been selected to directly evaluate human T cell response after protein/peptide immunization. Since T cell epitope sequences between human and mouse PHF20 are identical, the use of these mice and breast cancer cell line E0771-A2 (expressing HLA-A2 molecule) is well suited for preclinical tumor models. Immunization of tumor-bearing HLA-A2 and NSG-A2 (NOD SCID IL2rgama−/−:HLA-A2.1) Tg mice with PHF20 peptides activates antigen-specific CD8⁺ T cells, leading to a potent T-cell mediated responses against breast cancer. The following experiments are performed to determine whether immunization of these mice with DCs pulsed with PHF20 peptides is sufficient to inhibit tumor growth. Although both HLA-A2 and NSG-A2 mice can be used for inducing T cell response, HLA-A2 Tg mice are used as an example to illustrate our experimental design in this section.

Therapeutic Tumor Model in HLA-A2 Mice:

Since cancer patients have growing tumors, it is important to determine whether PHF20 peptide vaccination can generate therapeutic antitumor immunity. The following studies are performed in a therapeutic tumor model.

(a) Preparation of DCs Pulsed with PHF20 Peptides for Immunization:

DCs are prepared from HLA-A2 Tg mice. DCs are collected on day 7 and pulsed with PHF20 peptides or other control peptides at a peptide concentration of 10 μM for 90 min. After three washes with PBS, DCs/peptides are ready for use in immunization. Three vaccination groups are tested: (1) DC/PHF20 peptide. (2) DC/control peptide, and (3) DC/PBS.

(b) Immunization and Challenge with Breast E0771-A2 Tumor Cells:

E0771-A2 breast tumor cells (5×10⁵/mouse) are subcutaneously injected into HLA-A2 Tg mice (6 per group) on day 0. These tumor-bearing mice are immunized by i.v. injection of 3×10⁵ DCs loaded with either PHF20 or control peptides on day 5. Tumor growth is monitored using calipers every 2 days. Differences in tumor growth inhibition among groups are statistically analyzed.

(c) Analysis of CD8⁺ T Cell Response:

To assess the function of CD8⁺ T cells, splenocytes from the immunized mice or from those with tumor regression are collected. Lymph nodes from three mice per group are pooled, and single cell suspensions are prepared by passing the samples through nylon mesh followed by centrifugation on a Ficoll gradient. The phenotypes of these cells are analyzed by FACS after staining with anti-CD4 and anti-CD8 antibodies. Antigen-specific T cell function is tested by different methods.

i) Cytokine Profile Analysis:

GM-CSF. IFN-γ, IL-2, IL-4, IL-10, TGF-β and IL-17 secretion from CD8⁺ T is analyzed by ELISA using the supernatants of activated T cells for 16 hr.

ii) ELISPOT Assay:

A detailed protocol for ELISPOT assay has been described in a recent study of antigen-specific T cells (Fu et al., 2004). Colored spots are counted using a microscope.

iii) Tetramer Assay:

HLA-A2/PHF20 tetramer is used to detect antigen-specific CD8⁺ T cells, as we did for TRP2-specific CD8⁺ T cells, as previously described (Fu et al., 2004).

Toxicity Study of PHF20 Peptide Immunization.

No toxic effects were observed from other peptide vaccines in cancer patients, the possibility of inducing autoimmune responses remains. The acute and chronic toxicities of PHF20 peptides in HLA-A2 Tg mice is evaluated, which C57BL/6 mice serving as a control.

Doses:

Two strategies for peptide delivery are selected. One is that DCs loaded with PHF20 peptide in different concentrations (0, 3, 30, 100 mg/mL) are injected intravenously. The second one is to inject PHF20 peptides subcutaneously. Based on experience with human NY-ESO-1 peptide phase I clinical trial, the peptide dose is 1 mg/per injection, which is equal to 0.28 μg/mouse. Thus, for acute toxicity study, we will use 10- and 100-fold higher concentrations of peptide (i.e., 3 and 30 μg/mouse) are used. All groups (10 HLA-A2 mice each) are i.v. injected with different doses of PHF20 or control peptide (for example, 0, 3, 30 and 60 μg/mouse). C57BL/6 mice are used as a specificity control for PHF20 peptide. For testing acute toxicity, mice receive one injection daily for 6 consecutive days; otherwise, they receive one injection every week for 6 months to evaluate chronic toxicity of the peptide.

Animal Behavior and Weight:

Each group is evaluated daily for mortality, behavior, and signs of pain or distress. Food consumption and body weights are monitored weekly.

Clinical Pathology:

In acute toxicity studies, blood is drawn for hematology (CBC) and serum chemistry assessments prior to the first dose (day 0), and then once a week (0, 7, 14, 21 and 28 days). For determining chronic toxicity, blood is collected every 2 weeks (day 0, 14, 28, 42 and 56) for the first 2 months, and then every 30 days for the next 4 months, for a total of nine blood samples. Hematology and serum chemistry tests are conducted. Serum is analyzed for the production of inflammatory cytokines such as IL-1 and IL-6, tested for autoantibody production for nucleolar antigens (ANA) and dsDNA. Necropsy with full gross and microscopic pathology is performed by the Pathology Core at TMHRI.

Statistical Analysis:

All of the data generated are analyzed in a standard manner, as follows. Baseline measurements of each endpoint are made and compared with the corresponding values for the control group. Descriptive statistics, including means, standard deviations, medians, and ranges, are computed for each group. Pairwise comparisons are performed with techniques that control for the experiment-wise error rate. For animal experiments, sample sizes of 6 per group are adequate to achieve 81% power to detect a difference between groups at a significance level (alpha) of 0.01, using a two-sided two-sample t-test. Differences in tumor volumes are evaluated with Independent Samples t-tests or Mann-Whitney U tests at the last end point. Repeated Measure ANOVA are used to test the difference in growth over time. Sample size calculations are done with PASS 2002 (NCSS and PASS, Kaysville, Utah), and all analyses are performed with the SPSS 12.0 software package (SPSS Inc., Chicago, Ill.).

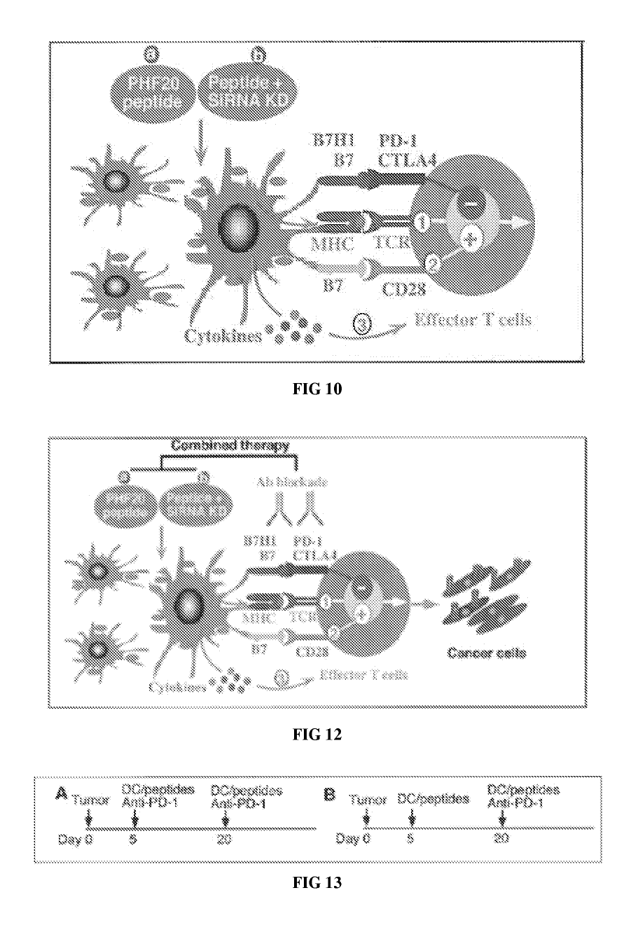

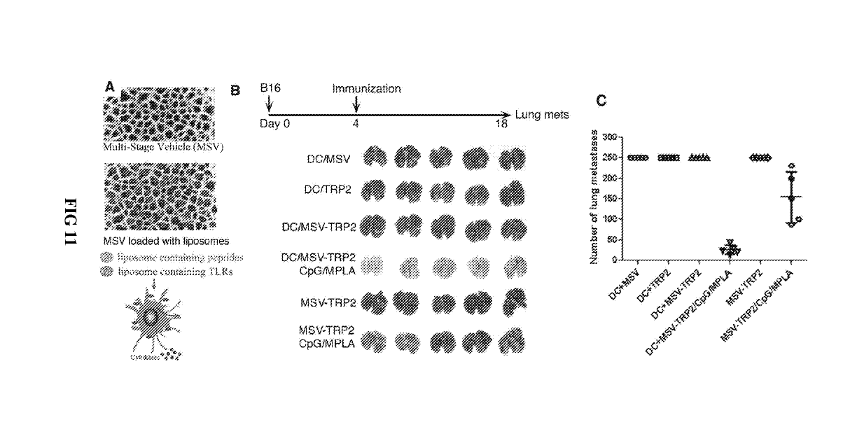

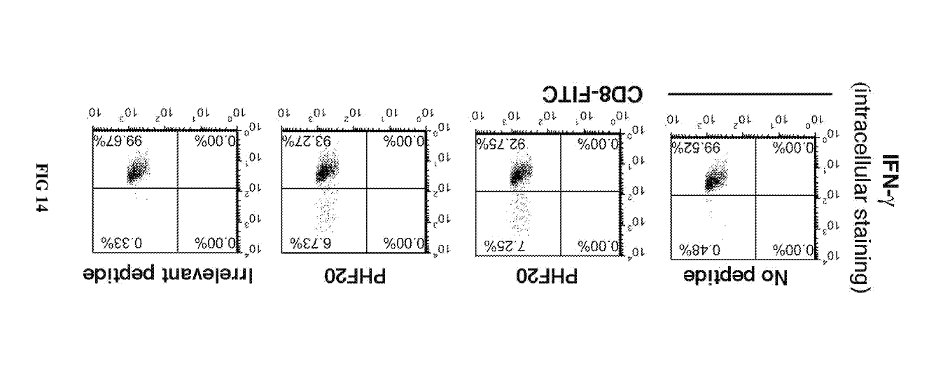

DCs play a critical role in the induction of immunity or tolerance, but one key to endowing the capacity of DCs to induce antigen-specific immune response is to knock down negative regulatory molecules in DCs, besides stimulation with cancer antigenic peptides and TLR7/9 agonists. Thus, knockdown of negative regulatory molecules releases DCs to achieve their maximal capacity to stimulate T cell response (FIG. 10). To effectively deliver peptides and small molecules (TLR ligands and siRNA), a multistage nanotechnology delivery system (Ferrari, 2005; Grattoni et al., 2011) is used.

Several negative regulators, including NLRC5, NLRX1 and NLRP4 have been identified that potently inhibit NFκB activation and type I IFN signaling. Knockdown of these negative regulators enhance innate immune response and cytokine production. Silencing of these negative regulators endows DCs with the unique capacity to induce robust antigen-specific immune responses. Knockdown of NLRX1 is used to illustrate the overall strategy (FIG. 10), with knockdown of A20 in DCs serving as a positive control.

a) Generation of NLRX1-KD DCs:

NLRX1-KD DCs are directly isolated from NLRX1-KD×HLA-A2 or HLA-A2 transgenic mice. Alternatively, DCs from HLA-A2 mice are transduced with NLRX1 specific lentivirus-shRNA, as previously described (Fu et al., 2004).

b) Tumor Injection and DC/Peptides Immunization in Therapeutic Tumor Models:

HLA-A2 mice (6 per group) are subcutaneously injected with E0771-A2 breast tumor cells on day 0. Wild-type DCs and NLRX1-KD DCs are loaded with PHF20 peptide or control peptide. After three washes with PBS, the DCs/peptides are ready for use in immunization. DC/peptide vaccination may begin on day 5 post tumor inoculation with the following experimental groups: 1) DC/PHF20 peptide: 2) DC/control peptide; 3) NLRX1-KD DC/PHF20 peptide: and 4) NLRX1-KD DC/control peptide. Tumor growth is monitored and measured with a digital caliper every 2 days. Antigen-specific T cell function is evaluated by intracellular staining, ELISA and tetramer analyses, as previously described (Ea et al., 2006).

Surface chemical modifications have been optimized such that the particles can evade the biological barriers, and enrich the tumor tissue. Using photolithographic synthesis protocols, we can produce first-stage particles of essentially any size and shape, so that the entire space within the design maps can be realized. The second stage nanoparticles could be liposomes and micelles incorporated with small molecule drugs such as peptides, TLR agonists and siRNA. Therefore, we intend to incorporate TRP2 peptides, TLR7/9 agonists, Poly-G3 OND and/or siRNA into nanoliposomes. These nanoliposomes are then loaded into mesoporous silicon (MSV/liposomes) (FIG. 1IA). MSV/liposomes can directly be loaded onto DCs in vitro and i.v. injected into mice. An alternative approach is to directly inject these MSV/liposomes into mice.

To demonstrate the power of MSV-delivery system, we recently performed experiments using B16 tumor and TRP-2 peptide (which can be presented by K^{b} in mice and HLA-A2 in humans), and found that mice immunized with DCs loaded with MSV-TRP-2 (MSV/liposomes containing TRP-2 peptide) rejected established B16 tumor growth in lungs. By contrast, DC/MSV, DC/TRP-2 or MSV-TRP-2 immunization failed to reject tumor growth (FIG. 1I). These results show that both DCs and MSV/liposome delivery system are needed to achieve a potent antitumor immunity. Combined use of cancer peptides, TLR ligands and/or siRNA via nanotechnology-based MSV/liposome delivery system markedly enhance the therapeutic efficacy of cancer immunotherapy and minimize side effects. Cancer antigenic peptides, TLR7/9 agonists and/or siRNA can be packed into the same or different nanoparticles.

c) Preparation of DCs Loaded with Nanoparticles:

DCs will be prepared and collected on day 7 and loaded with MSV/liposome containing PHF20 peptide (MSV/PHF20), TLR7/9 ligands and/or siRNA for negative regulatory molecules as previously described (Wang et al., 2002). After three washes with PBS, the DCs/nanoparticles are ready for use in immunization.

d) Immunization and Monitoring of Tumor Growth:

To investigate the combined therapeutic effect of DCs loaded with MSV/liposome containing PHF20 peptides, TLR7/9 agonists, and/or siRNA for negative regulatory molecules such as NLRX1 on the established tumor, E0771-A2 tumor cells (5×10⁵/mouse) are subcutaneously injected into HLA-A2 Tg mice (6 per group) on day 0. These mice are immunized on day 5 by i.v. injection of 3×10⁵ DCs loaded with 1) MSV/PHF20; 2) MSV/control peptides: 3) MSV/PHF20⁺ TLR7/9 agonists; 4) MSV/PHF20⁺ TLR7/9 agonists+NLRX1 shRNA. Tumor growth is monitored every 2 days.

B7-H1 (also known as CD274, PD-LI) is a critical negative regulator of T cell activation, which was found to be constitutively expressed by the majority of freshly isolated human cancer samples. B7-H1 is a major ligand for its receptor, programmed death-1 (PD-1), to deliver an inhibitory signal to T cells, leading to suppression of immune responses (Samstein et al., 2012; Woo et al., 2012). The mechanisms underlying B7-H1/PD-1 mediated suppression include induction of apoptosis, anergy and exhaustion of recently activated effector T cells (Woo et al., 2012; Zou and Chen, 2008). Therefore, antitumor T cell immunity could not execute its function due to the presence of B7-H1/PD-1 suppression in the tumor microenvironment. Upregulation of PD-1 have been observed in the exhausted CD8⁺ T cells from virally infected patients and cancer patients (Darce et al., 2012: Samstein et al., 2012; Matsuzaki et al., 2010). This explains, at least in part, why the presence of peripheral T cell response and the presence of tumor-infiltrating lymphocytes (TIL) do not lead to the regression of cancer (Taube et al., 2012). Recent clinical trials with anti-PD-1 antibody show durable tumor regression with objective clinical response rate of 18-28% for lung cancer, melanoma and renal-cell cancer.

Given that multiple suppressive mechanisms are operating to potently inhibit both CD4⁺ and CD8⁺ T cell responses, we hypothesize that blockade of PD-1 negative signaling in T cell activation will enhance antitumor immunity. Immunization of mice with PHF20 peptides will induce antigen-specific immunity, but may not be sufficient to destroy tumor cells. Increasing evidence indicates that blockade of negative signaling or checkpoint (CTLA-4 and PD-1) pathways are required for enhancing antitumor T cell response. Thus, optimal antitumor immunity may be generated by PHF20 peptide vaccination to produce antigen-specific T cells, in combination with anti-PD-1 or anti-PD-LI blockade, thus resulting in potent therapeutic antitumor immunity (FIG. 12).

PHF20 peptide induced antitumor immunity is enhanced by anti-PD-1 blockade. Humanized NSG-A2 mice are immunized with DC/PHF20 peptide in the presence or absence of anti-human PD-1 (anti-PD-1) antibody. Experimental procedures are outlined below.

i) Preparation of Humanized NSG-A2 Mice:

Humanized NSG-A2 mice are used to demonstrate the feasibility and effectiveness of PHF20 peptide vaccination in the induction potent antitumor immunity. The NSG-A2 (NOD SCID IL2rgama−/−:HLA-A2.1) mice have been used a useful preclinical model for viral infection and cancer study. Humanized NSG-A2 mice are generated using a previously described protocol. Briefly, 2-5-d-old NSG mice are irradiated with 100 cGy and injected intrahepatically with 1-3×10⁵ CD34⁺ HSCs 6 hr after irradiation. The mice are bled 10-12 wk after engraftment, and peripheral lymphocytes analyzed by FACS.

ii) Preparation of DCs Loaded with PHF20 Peptides:

Human DCs are prepared from HLA-A2+PBMCs and collected on day 7 and loaded with PHF20 peptide or a control peptide, as previously described 3×. After three washes with PBS, the DCs/PHF20 peptides are ready for use in immunization. Alternatively, DCs are loaded with MSV/liposome containing PHF20 peptide (MSV/PHF20), TLR7/9 ligands and/or siRNA for immunization.

iii) Immunization and Monitoring of Tumor Growth:

We subcutaneously inject HLA-A2 human breast tumor cell line MCF-7 (5×10⁵/mouse) into humanized NGS-A2 Tg mice (6 per group) on day 0. These tumor-bearing mice are immunized by i.v. injection of 3×10⁵ DCs loaded with either PHF20 or control peptides on day 5. Since anti-PD-1 blockade could be done at the same time as DC/PHF20 vaccination or after DC/peptide vaccination, we perform experiments to determine the optimal combination schedules. As depicted in FIG. 13, the following treatment groups are included:

A. Vaccination Plus Anti-PD-1 Blockade Schedule

1) on day 5, DCs/PHF20 peptide immunization only, and on day 20 DCs/PHF20 peptides
2) on day 5, DCs/PHF20 peptides-control antibody, and day 20 DCs/PHF20 peptides+control antibody
3) on day 5, DCs/PHF20 peptides+anti-PD-1 antibody, and day 20 DCs/PHF20 peptides+anti-PD-1

B. Priming and Boosting Plus Anti-PD-1 Blockade Schedule

1) on day 5, DCs/PHF20 peptides, and on day 20 DCs/PHF20 peptides
2) on day 5. DCs/PHF20 peptides, and on day 20 DCs/PHF20 peptides+control antibody
3) on day 5, DCs/PHF20 peptides, and on day 20 DCs/PHF20 peptides+anti-PD-1 antibody Tumor growth will be monitored every 2 days. Differences in tumor growth inhibition among groups will be statistically analyzed.

Detection and Induction of Antigen-Specific T Cells:

To correlate T cell responses with antitumor immunity, we use PHF20-peptide tetramers and ELISPOT to monitor CD8⁺ T cell responses. We also use ELISA to measure different cytokine production by T cells.

The present example characterizes the biological potency and toxicity of DC/PHF20 peptide stimulation or vaccination in the induction of antigen-specific T cell response and antitumor immunity, and has identified the most promising strategies to generate strong immune response by knockdown of negative regulators in DCs. The robust antitumor immunity is achieved by combining PHF20 peptide vaccines with anti-PD-1 blockade. This study provides a path for the development of PHF20-based anti-PD-1 enhanced immunotherapy vaccines/drugs for the treatment of cancer, and in particular, metastatic breast cancer.

Generation of PHF20-specific T cells in HLA-A2 transgenic mice: To demonstrate the possibility that immunization of HLA-A2 transgenic (Tg) mice with DC/PHF20 peptide can also induce antigen-specific T cell response, we performed experiments and found that immunization of HLA-A2 Tg mice with DCs loaded with PHF20 peptide generated strong antigen-specific T cells response that specifically recognized PHF20 peptide (WQFNLLTHV), but an irrelevant peptide (FIG. 14).

HLA-A2 Tg mice were immunized with DC/PHF20 peptide. Eight days later, T cells were isolated from splenocytes and tested for their ability to recognize PHF20 or irrelevant peptide. T cells were stained with anti-CD8-FITC and then intracellular stained with anti-IFN-γ-PE. FACS analysis was performed after gating on CD8⁺ T cell population.

### Examples

The following examples are included to demonstrate illustrative embodiments of the invention. It should be appreciated by those of ordinary skill in the art that the techniques disclosed in these examples represent techniques discovered to function well in the practice of the invention, and thus can be considered to constitute preferred modes for its practice. However, those of ordinary skill in the art should, in light of the present disclosure appreciate that many changes can be made in the specific embodiments which are disclosed and still obtain a like or similar result without departing from the spirit and scope of the invention.

Jmjd3 Negatively Regulates Reprogramming Through Histone Demethylase Activity-Dependent and -Independent Pathways

In this example, Jmjd3 has been identified as a potent negative regulator of somatic cell reprogramming in screening studies of a panel of histone-modifying proteins. Knockdown or ablation of Jmjd3 enhanced the efficiency and kinetics of reprogramming, apparently by dual mechanisms: 1) Jmjd3 partially inhibits iPSC reprogramming by promoting cell senescence through upregulation of p21 and Jnk4a, and 2) Jmjd3 targets PHF20 (plant homeodomain finger protein 20) for ubiquitination and proteasomal degradation via the E3 ubiquintin ligase Trim26 in a demethylase activity-independent manner. Knockdown or ablation of PHF20 blocks the reactivation of endogenous Oct′ expression, thus leading to partially programmed cells. These results implicate the Jmjd3-PHF20 axis as a key pathway in somatic cell reprogramming, and provide novel insights into the molecular mechanisms used by Jmjd3 to impede efficient reprogramming.

Experimental Procedures

Mice

Rosa-rtTA Tet-O-Oct4 transgenic mice were purchased from the Jackson Laboratories (strain 006911). Tet-O-Myc transgenic mice were obtained from Baylor College of Medicine. Ezh2ff mice were obtained from The University of North Carolina (UNC)-Mutant Mouse Regional Resource Center (MMRRC) (Su et al., 2003). ERT Cre transgenic mice were purchased from the Jackson Laboratories (strain 004847). PHF20 knockout mice were obtained from M.D. Anderson Cancer Center (Badeaux et al., 2012). Jmjd3 was targeted by deletion of exon 15-21 using a Cre-LoxP system. Jmjd3 globally deleted by crossing Jmjdf^{f} mice with Hprt-Cre mice (Jackson Laboratories, strain 004302). Tet-O-Sox2, -Klf4 and -PHF20 transgenic mice were generated at Baylor College of Medicine. Two independent transgenic lines for each gene were established and maintained by crossing two founders with C57BL/6 mice. These mice were crossed to rtTA-expressing Tet-O-Oct4 and TetO-Myc transgenic mice to generate quintuple-transgenic lines. MEFs expressing rtTA and Tet-OOct4, Sox2, Klf4 and Myc were established from transgenic mice. All mice were maintained in a pathogen-free animal facility. All animal studies were performed using approved protocols.

Cell Culture

mESCs and miPSCs were cultured in mESC medium (DMEM with 15% FBS, 1 mM L-glutamine (Invitrogen), 1% nonessential amino acids (Invitrogen), 0.1 mM 1-mercaptoethanol (Sigma) and 1,000 U ml-1 LIF (Santa cruz)) on irradiated feeder cells.

MEFs were isolated by trypsin digestion of midgestation (E13.5) embryos followed by culture in fibroblast medium (DMEM with 10% FBS, 1 mM L-glutamine, 1% nonessential amino acids and 0.1 mM β-mercaptoethanol), hiPSC culture medium consists of DMEM/F12 with 20% Knockout Serum Replacement (Invitrogen), 1 mM L-glutamine, 0.1 mM mercaptoethanol, 1% non-essential amino acid solution, and 10 ng/mL of FGF2 (Invitrogen).

Lentivirus Transduction

All lentiviral particles were generated as previously described (Peng et al., 2005): 293T cells cultured on T175 flasks were transfected with a lentiviral vector expressing shRNA/cDNA (22.5 μg) together with the packaging plasmids VSV-G (10 μg) and Δ8.9 (15 μg) using lipofectamine 2000 (Invitrogen) transfection reagent. Viral supernatants were collected at 48 hr after transfection, yielding a total of ˜35 mL of supernatants per virus. Viral supernatants were further concentrated by ˜200-fold using ultracentrifugation at 25,000 rpm for 2 hr at 4° C. and resuspension in 175 μL of PBS. The MEFs were infected concentrated virus with polybrene (8 g/mL: Sigma). Typically, more than 90% of cells were successfully transduced using this methodology as judged by a GFP cDNA transduction. The lentiviral shRNAs information is shown in Table 2.

Cloning of the Full-Length Jmjd3 and PHF20 cDNAs and Various Mutants

To clone the full-length Jmjd3 cDNA, total RNA was isolated and Jmjd3 cDNA fragments were amplified by PCR. The 5-kb PCR product containing the Jmjd3 was cloned into the HA- or Flag-tagged pcDNA3.1 vector. Truncated deletion mutants were generated by performing PCR with different primers. A similar strategy was used to clone the full-length and truncates of PHF20. Jmjd3 H1390A mutation was generated using QuikChange II XL site-directed mutagenesis kit (Agilent technologies). All the cDNAs were sequenced to confirm that their sequences are identical to the published ones in the database.

Isolation of ICM and Establishment of ESC Lines

Blastocysts were isolated from PHF20^{+/-} intercrossed pregnant females at E3.5 day and cultured on the gelatin-coated 24-well plates with ESC culture medium. The growth of ICM were monitored and recorded daily. At day 4, ICM were staining with AP-kit. For establishment of the ESC lines, blastocysts at E3.5 day were cultured on 24-well plates with feeder cells in ESC-medium. At day 8, ESCs were isolated from ICM and further grown on feeder cells. These ESCs were continually passaged to P3.

Generation of Chimeric Mice

Fully reprogrammed iPSCs were microinjected into Balb/c blastocysts for chimeric mice; chimeric mice could be identified by coat color.

Bisulfite Genomic Sequencing Assay

Bisulfite conversion was performed using the Epitect Bisulfite Kit (QIAGEN). Molecules were cloned using the Topo TA Cloning Kit (Invitrogen), according to the manufacturers' instructions.

Jmjd3 Demethylase Activity Assay

293T cells were transfected with HA-Jmjd3 or various HA-Jnjd3 mutants. Nuclear lysates were collected after 48 h transfection. The Utx/Jmjd3 H3K27me3 demethylase activity detection Kit (Epigentek) was used to determine Jmjd3 H3K27me3 demethylase activity.

Immunofluorescence Staining

The cells were cultured on the pretreated cover slips, fixed with 4% PFA and permeabilized with 0.5% Triton X-100. The cells were then stained with primary antibodies to Oct4 (Santa Cruz), SSEA-1 (Abeam) or HA, followed by staining with the respective secondary antibodies conjugated to Texas Red. Nuclei were counterstained with DAPI (Invitrogen). Cells were imaged using a Leica DMI4000B inverted fluorescence microscope equipped with a C350FX camera.

Alkaline Phosphatase Staining

The Alkaline Phosphatase Detection Kit (Vector lab) was used to determine alkaline phosphatase activity according to the manufacturer's instructions.

Immunoprecipitation (IP), Immunoblot and Ubiquitination Analyses

Cells were lysed in low salt lysis buffer or RIPA buffer containing protease inhibitors. Samples were centrifuged at 10,000×g for 10 min and the supernatants were added to a 40-μL anti-HA gel or anti-Flag M2 affinity gel, as previous described (Cui et al., 2010). The samples were IP with specific antibodies over night at 4° C. The beads were then washed five times, eluted with 3×SDS/PAGE loading buffer and boiled for western blot. Endogenous co-IP was performed as described by using antibodies specific for anti-PHF20 (Cell Signaling Technology) followed by incubation with the immobilized protein A/G (Sigma). Chemiluminescent HRP substrate (Millipore) was used for protein detection. For analysis of PHF20 ubiquitination. HEK293T cells were transfected with PHF20, Jmjd3, Trim26 with or without WT ubiquitin or ubiquitin mutants containing only one lysine at position 48 (K48) or 63 (K63). Thirty-six hours after transfection, cell lysates were immunoprecipitated with indicated antibodies, including anti-PHF20 and anti-Flag antibody (Sigma), followed by immunoblot analysis with anti-ubiquitin or anti-K48 ubiquitin antibody for the detection of ubiquitination of PHF20.

Chromatin Immunoprecipitation (ChIP)-PCR and ChIP-Seq Analyses

ChIP assay was performed according to the Imprint Ultra Chromatin Immunoprecipitation Kit manual (Sigma). Briefly, ESCs and iPSCs were grown to an approximate final count of 1-5×10⁷ cells for each reaction. Cells were cross-linked with 1% formaldehyde solution for 10 min at room temperature and quenched with 0.125 M glycine. Cells were rinsed twice with 1×PBS. Cells were resuspended, lysed, and sonicated to solubilize and shear crosslinked DNA. The resulting chromatin extract was incubated overnight at 4° C. with 10 □g antibody. Next day, each sample was added 15 □L blocked beads and then incubated at 4° C. for 1 hr. Beads were washed 5 times with RIPA buffer. The complexes were eluted from beads in elution buffer by heating at 65° C. Input DNA (reserved from sonication) was concurrently treated for crosslink reversal. DNA were treated with RNaseA, proteinase K and purified. Primary antibodies used for IP were: PHF20 (Cell Signaling Technology), Wdr5 (Bethyl), mouse/rabbit IgG and RNA Polymerase II (Sigma). Relative Fold enrichments were calculated by determining the immunoprecipitation efficiency (ratios of the amount of immunoprecipitated DNA to that of the input sample). For ChIP-Seq analysis, a total of 30 ng of immunoprecipitated DNA fragments was used for the ChIP-Seq library construction. Illumina sequencing was performed. Sequencing reads from PHF20, Wdr5 and Polymerase II-pulled down ChIP-Seq libraries were aligned to the mouse mm8 genome using ELAND software. The statistical significance of the fold change was assessed using the MA-plot-based method (Wang et al., 2010).

ChIP-Seq libraries were prepared using standard protocols (available: www.illumina.com). The resulting libraries were sequenced on an Illumina Miseq instrument in two successive runs, and output pooled for each sample for analysis. The resulting sequence output (bases 2-42) were aligned to mouse genome version mm9 using bowtie 0.12.7 (Langmead et al., 2009). Bound peaks were analyzed using QuEST2.4 (Valouev et al., 2008), with standard parameters and specified enrichment of n-bold. The resulting genome-wide binding data were analyzed with utilities in the Cistrome portal (Liu et al., 2011). Genome-wide binding patterns were analyzed with CEAS (Shin et al., 2009). Over-represented transcription factor binding motifs were annotated using the seq position to query mouse or human motifs in the TRANSFAC database (Matys et al., 2006). TSS-proximal binding events were analyzed with the Genomatix software suite (http://www.genomatix.com). The bound and unbound genes were based on the significance of enrichment. Gene ontological analysis of proximal binding events was performed using web based bioinformatics database (http://david.abcc.ncifcrf.gov/).

Real-Time Quantitative PCR (Real-Time PCR)

Complementary DNA was generated from the total RNA of 293T, MEF and iPS cells with SuperScript II Reverse Transcriptase (Invitrogen), using oligo (dT) as a primer. Gene transcripts were quantified by real-time PCR with SYBR Green real-time PCR SuperMix for the ABI PRISM Instrument (Invitrogen) in an ABI Prism 7000 system (Applied Biosystems). All of the values of the target gene expression level were normalized to □-actin. The primers used for real-time PCR are listed in Table 1.

Knockdown of PHF20 During Reprogramming

To determine the function of PHF20 in different reprogramming stages, PHF20 was knocked down in different time points. Tet-O-4F M2-11 MEFs were seeded on feeder cells on day −1, and then transduced them with PHF20-specific lentiviras-based constitutively expressing shRNA on day 0. Culture medium (containing viruses) was exchanged with fresh ES medium with Dox. For knockdown at other time points, cells were infected with PHF20-specific or control lentiviral shRNA in ES medium with Dox on day 4, 8 or 12, as indicated. The infected cells were maintained in ES medium with Dox, and AP⁺ colonies were counted on day 14.

Generation of iPSCs from MEFs and Tet-O-4F MEFs

Mouse iPSCs were generated as previously described (Takahashi et al., 2007a) with some minor modifications. Briefly, MEFs (1-8×10⁴/well) were seeded on irradiated-MEFs in a 6-well plate. On the next day, the cells were transduced with an equal amount of lentiviruses expressing the four factors and rtTA. The following day, transduced-MEFs were cultured with mESC medium containing 2 μg/mL Dox for 14 days. Tet-O-4F MEFs were used to generate iPSCs by treating MEFs with Dox in mESC medium. The efficiency of iPSC formation as calculated based on the number of AP positive iPSC colonies and the initial cell number of seeded MEFs. Human iPSCs were generated as previously described (Park et al., 2008).

Screening for Identification of Epigenetic Factors for Modulating Reprogramming Using Tet-O-4F MEFs and shRNA Knockdown

Tet-O-4F transgenic MEF cells were transduced with lentiviral shRNA-specific for 15 epigenetic factors and then reseeded on irradiated feeder cells at the desired density. The next day, mESC medium containing 2 μg/mL Dox was added and replenished every day. The colonies were stained for AP activity on Days 12-14, and lentiviral particles were generated and concentrated, as previously described (Peng et al., 2005).

Real-Time Quantitative PCR and Immunoblotting

Total RNA was Trizol (Invitrogen) extracted, column purified, and reverse transcribed using SuperScript0 II Reverse Transcriptase kit (Invitrogen), as previously described (Cui et al., 2010). For ChIP-qPCR analysis, 1 ng ChIP-DNA was used for each PCR. All qPCR analyses were performed with SYBR Green (Applied Biosystems). To obtain whole-cell protein extracts for immunoblotting analysis, cells were lysed with low salt lysis buffer or RIPA buffer. Primer sequences and antibodies are described in Table 1.

Co-IP and ChIP Assay

The cells were lysed in low salt lysis buffer, incubated overnight with 5 μg antibody, and captured with Protein A/G beads, as previously described (Cui et al., 2010). Immunoprecipitants were eluted by boiling in loading buffer. 10 μL was used for each immunoblot with 2% whole cell lysates. Epitope tagged co-IP in 293T cells was performed with Flag, HA or Myc antibody in low salt lysis buffer. ChIP assay was performed with Imprint Ultra Chromatin Immunoprecipitation kit (Sigma). Primer sequences and antibodies are described in Table 1.

Results

Identification of Jmjd3 as an Inhibitor of Reprogramming

Ectopic expression of four transcription factors (4F) in somatic cells can induce the generation of iPSCs (Okita et al., 2007; Takahashi et al., 2007b; Takahashi and Yamanaka, 2006), but this reprogramming strategy requires viral transduction of the requisite factors, leading to variable outcomes. To establish a simpler and inducible 4F-based method to generate iPSCs, transgenic mice expressing tetracycline (Tet)-O-inducible Sox2 and Klf4 were generated, which were then crossed with Tet-O-inducible Oct4 and Myc transgenic mice carrying rtTA-M2 reverse tetracycline transactivator (FIG. 1A). Mouse embryonic fibroblasts (MEFs) were generated from transgenic mice expressing Tet-O-Oct4, -Sox2, -Klf4 and -Myc genes as well as rtTA-M2 and tested for their ability to express 4F once they were treated with doxycycline (Dox). As shown in FIG. 1B, Oct4, Sox2, Klf4, and Myc proteins were readily detected by immunoblot analysis when the cells were treated with 2 μg/mL Dox for 24 hr. It was shown that these 4F-expressing MEFs (Tet-O-4F MEFs) could be efficiently reprogrammed to generate iPSCs in the presence of Dox (FIG. 1C). Withdrawal of Dox before or at day 8 markedly reduced AP⁺ colony formation, but there was no appreciable difference in AP iPSC colony number when Dox was withdrawn at day 10 or later using three different (WT, Tet-O-4F and Oct4-GFP) types of MEFs. These fully programmed iPSCs stained positively for AP, SSEA-1 and Nanog (FIG. 1D-FIG. 1G). These results are consistent with reports from other groups (Carey et al., 2010; Stadtfeld et al., 2010; Wernig et al., 2008), suggesting that Tet-O-4F MEF-based reprogramming would provide a reliable system to screen for epigenetic factors that either enhance or reduce the efficiency of reprogramming.

The inventors predicted that epigenetic factors involved in histone modification play critical roles in reactivating the expression of stem cell-enriched genes, including Oct4, Sox2 and Nanog, while shutting down the expression of cell lineage-specific differentiation genes, thus greatly increasing the efficiency of 4F-mediated reprogramming. To test this hypothesis, a panel of shRNAs with high knockdown efficiency (>70%) was screened against a subset of genes encoding histone methyltransferases or demethylases, and then transduced into Tet-O-4F MEFs. After three rounds of screening, it was shown that knockdown of the H3K27 methyltransferase Ezh2 and many histone demethylase genes, including Fbx110, Taridlb, Jaridld, Jarid2, Jmjdla, Jmjd2c and Utx, markedly decreased reprogramming efficiency (FIG. 1H). This was consistent with previous results that showed Fbx110, Ezh2. Jmjdla, Jmjd2c and Utx played a critical role in ESC renewal and iPSC reprogramming (Ezhkova et al., 2009; Loh et al., 2007; Mansour et al., 2012; Onder et al., 2012: Wang et al., 2011). By contrast, knockdown of Jmjd3 markedly increased the efficiency of 4F-mediated reprogramming, while its ectopic expression resulted in decreased reprogramming efficiency (FIG. 10). These findings suggest that Jmjd3 functions as a barrier to iPSC generation from somatic cells, even though many histone methyltransferases and demethylases are clearly required for this process. These unique features of Jmjd3 led to its selection for further study.

Jmjd3 Ablation Enhances the Efficiency and Kinetics of Reprogramming

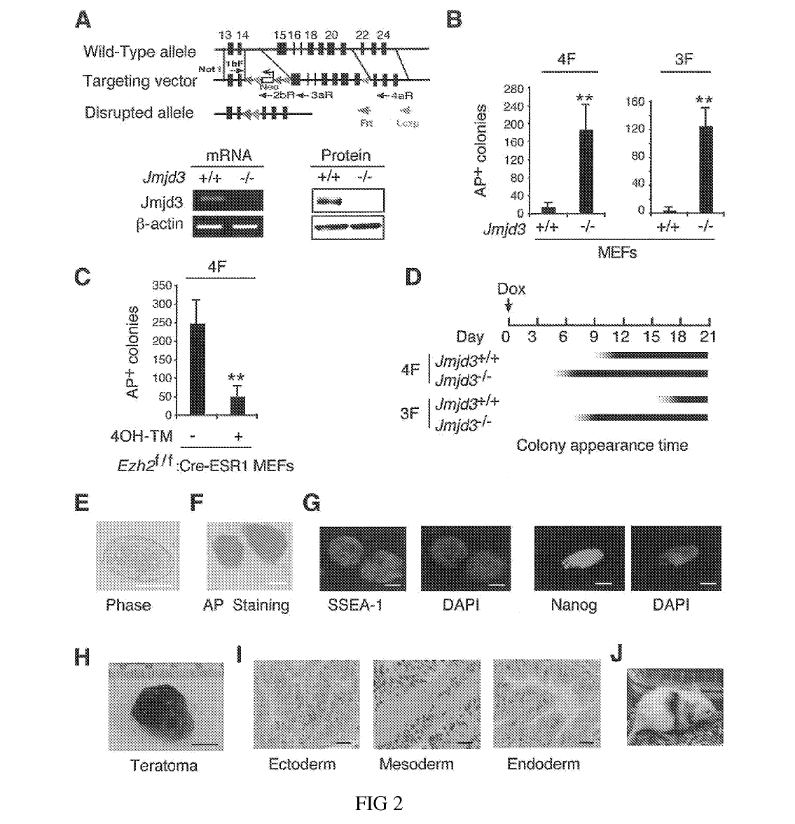

To further define the role of Jmjd3 in reprogramming, Jmjd3 knockout mice were generated by targeted deletion of exon 15-21 using a Cre-LoxP system (FIG. 2A). Mice in which Jmjd3 was globally deleted by crossing Jenjd3^{l} mice with those expressing the Cre recombinase gene driven by the hypoxanthine guanine phosphoribosyl transferase promoter (Hpri-Cre) died shortly after birth, with defects in lung and bone formation. RT-PCR and western blot analyses of Jmjd3-deficient MEFs showed that the expression of Jmjd3 was abrogated in Jmjd3-deficient MEFs, compared with WT controls (FIG. 2A). Consistent with results obtained by Jmjd3 knockdown, it was found that 4F-reprogrammed Jmjd3-deficient MEFs significantly more iPSC colonies than did WT MEFs (FIG. 2B). Robust reprogramming was also achieved with Jmjd3-deficient 3F-transduced MEFs (Oct4, Sox2 and Klf4), compared to the outcome with WT MEFs (FIG. 2B). By contrast, Ezh2-deficient MEFs, which were generated by treating Ezh2^{11,407}:Cre-ESR MEFs with tamoxifen, strikingly inhibited the efficiency of 4F-mediated reprogramming of MEFs (FIG. 2C), further confirming that Ezh2 is necessary for reprogramming. More importantly, it was found that AP⁴⁻ iPSC colonies appeared much earlier in Jmjd3-deficient MEFs than in WT MEFs, whether reprogramming was mediated with 3F or 4F (FIG. 2D), suggesting that Jmjd3 ablation markedly increases the kinetics and efficiency of reprogramming.

The iPSCs generated from Jmjd3-deficient MEFs showed characteristic ESC morphology and markers, e.g., positive immunological staining for AP, phosphatase, SSEA-1 and Nanog (FIG. 2E-FIG. 2G). They also formed teratomas comprising all three embryonic germ layers (ectoderm, mesoderm and endoderm) (FIG. 2H-FIG. 2I), and contributed to chimeras after injection into BALB/C host blastocysts (FIG. 2J). Thus, iPSCs generated from Jmjd3-deficient MEFs possess the same hallmarks of pluripotency as those derived from WT MEFs, indicating that loss of Jmjd3 enhances the efficiency and kinetics of iPSC reprogramming, supporting a negative regulatory role for this protein.

Jmjd3 Negatively Regulates Reprogramming of the Ink4a/Arf Locus

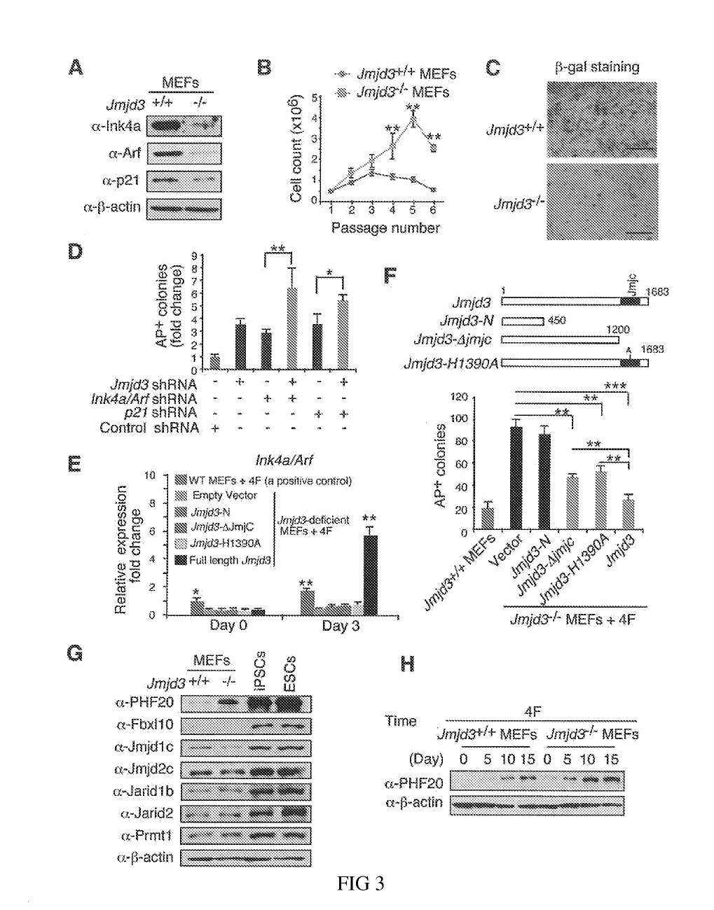

The inventors next asked how Jmjd3 ablation enhanced reprogramming. Jmjd3 expression was thought to increase the expression of Ink4a/Arf in MEFs by modifying H3K27 methylation in the promoter region of the Ink4a/arf locus through the demethylating activity of its Jumonji domain in the C-terminus (Agger et al., 2009; Barradas et al., 2009). Furthermore, the expression of several key molecules, including Ink4a, Arf and p21, play a critical role in cell growth arrest and senescence, and their deficiency reduces cell senescence while markedly increasing the efficiency of reprogramming (Hong et al., 2009; Kawamura et al., 2009; Li et al., 2009; Marion et al., 2009; Utikal et al., 2009). To assess the expression level of these molecules in Jmjd3-deficient MEFs, both RT-PCR and western blot analyses were performed. It was observed that Jmjd3 deletion markedly reduced the expression of Inklalibilf mRNA and protein, compared with findings in WT cells (FIG. 3A). p21 protein expression was also reduced in Jmjd3-deficient MEFs, although a difference in p21 mRNA was not evident between WT and Jmjd3-deficient MEFs (FIG. 3A). Thus, Jmjd3 deletion sharply reduces the expression of Ink4a and Arf proteins, and to a lesser extent, that of the p21 protein. These effects may in turn reduce cellular senescence and increase cell proliferation. Indeed, it was found that Jmjd3-deficient MEF cells grew faster than WT cells (FIG. 3B). Cellular senescence based on p-galactosidase 03-gal) staining in Jmjd3-deficient MEFs was also reduced, compared with results in WT MEFs (FIG. 3C). Although the Jmjd3-deficient MEFs underwent a senescence crisis after 5-7 passages, a short-term reduction of senescence and an increase of cell proliferation due to Jmjd3-deficiency may have contributed in a transient manner to the improved efficiency and kinetics of reprogramming in these MEFs.

To determine the extent to which downregulation of Ink4alArf and p21 accounts for the more efficient reprogramming in Jmjd3-deficient MEFs, the expression of these genes was knocked down with specific shRNAs and their effects on reprogramming in Tet-O-4F MEFs were assessed. Although knockdown of Jmjd3, Ink4a/Arf or p21 alone by shRNAs increased reprogramming efficiency (compared to that in MEFs transduced with a control shRNA), the efficiency nearly doubled with simultaneous knockdown of Jmjd3 and Ink4a/Arf or p21 (FIG. 3D) suggesting that Jmjd3 might have additional effects on reprogramming that do not overlap with those mediated by Ink4a, Arf and p21. Jmjd3-N (containing the N-terminal 450 aa), Jmjd3-AJmjC (containing a deletion in the catalytic Jumonji domain) and Jink13-111390A (containing a point mutation in the catalytic domain) constructs were made, all of which lack the H3K27me3 demethylase activity of H3K27 trimethylation. In experiments testing whether Jmjd3-mediated inhibition of reprogramming depends upon expression of Ink4a/Arf and p21, ectopic expression of full-length Jmjd3, but not Jmjd3-N, Jrnjd3-4JnajC or Jmjd3-H1390.4, in Jmjd3-deficient MEFs restored the expression of Ink4a/Arf (FIG. 3E) and almost completely inhibited reprogramming (FIG. 3F). Surprisingly, two Jmjd3 mutants (Jmjd3-AlmjC and Jmjd3-H1390A) that lacked H3K27 demethylase activity and failed to upregulate Ink4a/Arf expression were still capable of inhibiting reprogramming in Jmjd3-deficient MEFs. These results clearly indicate that Jmjd3 can modulate reprogramming through both demethylase activity-dependent and -independent pathways.

PHF20 is a Key Target of the Jmjd3 Protein

To search for the targets of Jmjd3, a comparative analysis of miRNA and mRNA gene expression was performed between WT and Jmjd3-deficient MEFs, but this study failed to identify any gene that could be responsible for the increased reprogramming efficiency in Jmjd3-deficient MEF cells. Hence, attention was focused on the expression levels of histone epigenetic factors, because they are critical in reprogramming somatic cells to an ESC-like state (Plath and Lowry, 2011; Stadtfeld and Hochedlinger. 2010). By comparing the expression of 59 genes that encode for histone modification proteins, 18 genes were identified that were markedly upregulated at the RNA level in iPSCs/ESCs, versus MEFs, and 11 genes that were upregulated between iPSCs/ESCs versus human fibroblasts. Comparison of expression patterns between fibroblasts and iPSCs/ESCs identified seven genes that were highly expressed in both mouse and human iPSCs/ESCs. Of these, only PHF20 (encoding the PHD finger protein 20, also called GLEA2) showed a marked increase of expression in Jmjd3 deficient MEFs, iPSCs and ESCs, versus WT MEFs (FIG. 3G); however, there was no appreciable difference in PHF20 mRNA expression between WT and Jmjd3-deficient MEFs.

Nor was there any appreciable difference in H31 (27 trimethylation between WT and Jmjd3-deficient MEFs or iPSCs. Furthermore, PHF20 was strongly expressed in testis, ovary and ES cells; weakly in placenta, lung, liver and muscle; and only slightly or not at all in other tissues. In time-course experiments to determine the expression pattern of PHF20 during reprogramming, a gradual increase of its expression in WT MEFs was found, which was accelerated in Jmjd3-deficient MEFs (FIG. 3H). These results suggested that the PHF20 protein was a key target of Jmjd3, and thus may play an important role in the renewal and maintenance of ESCs, iPSCs, or both.

Requirement for PHF20 Expression in the Maintenance and Reprogramming of ESCs and iPSCs

Because the PHF20 protein is abundantly expressed in both ESCs and iPSCs, its importance in the maintenance of these cell types was assessed. After knocking down PHF20 in ESCs with specific shRNAs that express GFP, evidence of differentiation was found, while ESCs transduced with control shRNA remained undifferentiated (FIG. 4A). Furthermore, RT-PCR and western blot analyses revealed that PHF20 expression in ESCs, like that of Oct4 and Nanog, was markedly reduced after withdrawal of leukemia-inhibiting factor (LIF) and addition of retinoic acid (RA) in the culture medium (FIG. 4B and FIG. 4C). Similar results were obtained with iPSCs. To determine whether stable ESC lines could be derived from WT and PHF20 knockout mice, it was shown that ESC lines could be readily generated from WT mice but not from PHF20 knockout mice. WT ESCs expressed AP, Nanog and Oct4 proteins, whereas cells from PHF20 knockout blastocysts did not, and differentiated rapidly into trophectoderm, based on downregulation of Oct4 and upregulation of Cdx2. Together, these data suggested that PHF20 was required for the generation and maintenance of both ESCs and iPSCs.

To further define the role of PHF20 in iPSC generation, the protein was knocked down in Tet-O-4F MEFs at different time points, and its ability to form iPSC colonies was examined. Knockdown of PHF20 in the early stages of reprogramming (i.e., at day 0 or 4) almost completely blocked iPSC generation, whereas in the intermediate or later stages (day 10 or 12) it led to a decreased (but still significant) inhibitory effect on the numbers of iPSCs formed (FIG. 4D). This finding was substantiated by use of PHF20 knockout MEFs isolated from PHF20 knockout mice, showing the loss of PHF20 expression at both the RNA and protein levels in MEFs (FIG. 4E). Reprogramming to iPSCs with either 3F or 4F was significantly inhibited in PHF20-deficient MEFs (FIG. 4F), and the few iPSC colonies that were generated from PHF20-deficient MEFs showed only partially reprogrammed iPSCs (FIG. 4G), suggesting that PHF20 is required for the efficient generation of fully reprogrammed iPSCs.

Results with MEFs suggest that Jmjd3 deletion enhances reprogramming, while PHF20 ablation inhibits it. This notion was further supported by studies using human fibroblasts for 4F-mediated reprogramming, in which that Jmjd3 knockdown enhanced reprogramming, while PHF20 knockdown blocked this process. To clarify how Jmjd3 and PHF20 reciprocally regulate reprogramming, Jnjd3/PHF20 single- or double-knockout MEFs were generated, and tested for their ability to regulate reprogramming. Both Jmjd3-deficient and Jmjd3/PHF20 double-knockout MEF cells grew faster than WT and PHF20-deficient cells, but no appreciable difference was observed in the growth between WT and PHF20-deficient cells. As expected, Jmjd3 deletion enhanced reprogramming, but PHF20 ablation inhibited this process (FIG. 4H). Remarkably, Jmjd3 deletion failed to improve reprogramming in Jmjd3 and PHF20 double-knockout MEFs (FIG. 4H), suggesting that the proliferative advantage of Jmjd3-deficient MEFs cannot overcome the failure of reprogramming in PHF20-deficient MEFs. Similar results were obtained when either Ink4a or p21 was knocked down in PHF20-deficient MEFs: that is, loss of each of these regulators increased reprogramming in WT MEFs, but failed to rescue defective reprogramming in PHF20 deficient MEFs (FIG. 41). Ectopic expression of PHF20, by contrast, restored reprogramming in PHF20-deficient MEFs (FIG. 4J), suggesting a requirement for expression of this gene in both WT and Jmjd3-deficient MEFs.

To further examine the ability of PHF20 expression to facilitate reprogramming, Tet-O-PHF20 MEFs were generated from rtT4:Tet-O-PHF20 transgenic mice and treated with Dox. This resulted in increased expression of PHF20, compared with findings in Dox-treated rtTAexpressing WT MEFs (FIG. 4K). More importantly, it was observed that Dox-induced expression of PHF20 in these cells led to a marked increase in the efficiency of 4F-mediated reprogramming, compared with findings in rtT4-expressing WT MEFs treated with Dox (FIG. 4K). Furthermore, overexpression of PHF20 could reverse the Jmjd3-mediated inhibition of reprogramming (FIG. 4L). The increased reprogramming efficiency in Tet-O-PHF20 MEFs was not due to cellular proliferative activity, because there was no appreciable difference in cell growth between WT and Tet-O-PHF20 MEFs, with or without Dox treatment. Instead, Dox-induced expression of PHF20 markedly blocked downregulation of Oct4, Sox2 and Nanog in iPSCs and thus their differentiation after LIF withdrawal. Nonetheless, PHF20 overexpression could not substitute for any of the 4F. These results indicate as essential requirement for PHF20 in somatic cell reprogramming, although its increased expression cannot substitute for any of the four established factors.

Jmjd3 Interacts with PHF20 and Mediates its Proteasomal Degradation

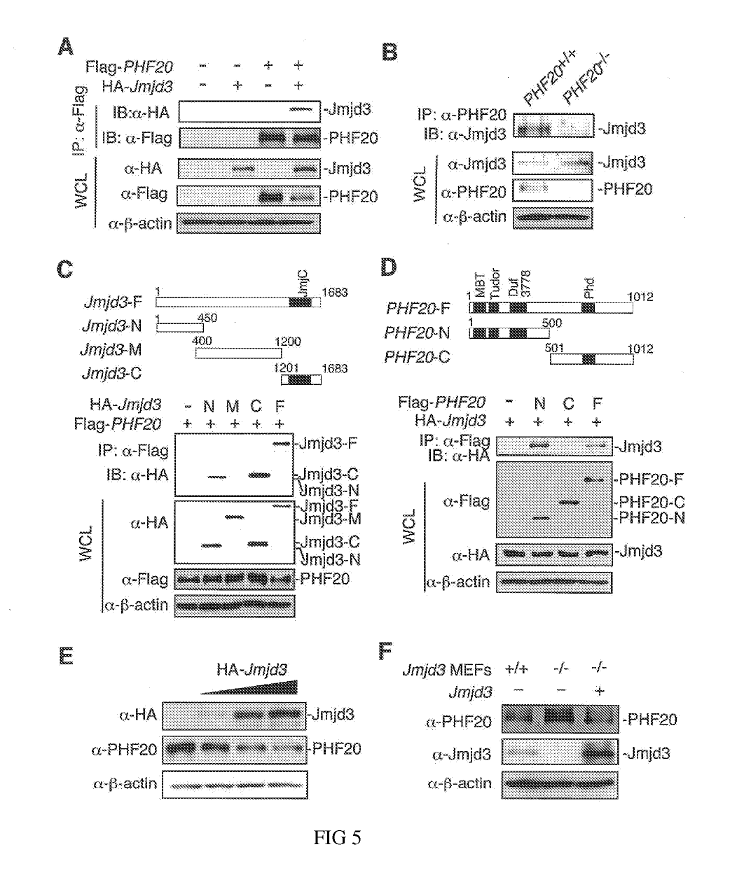

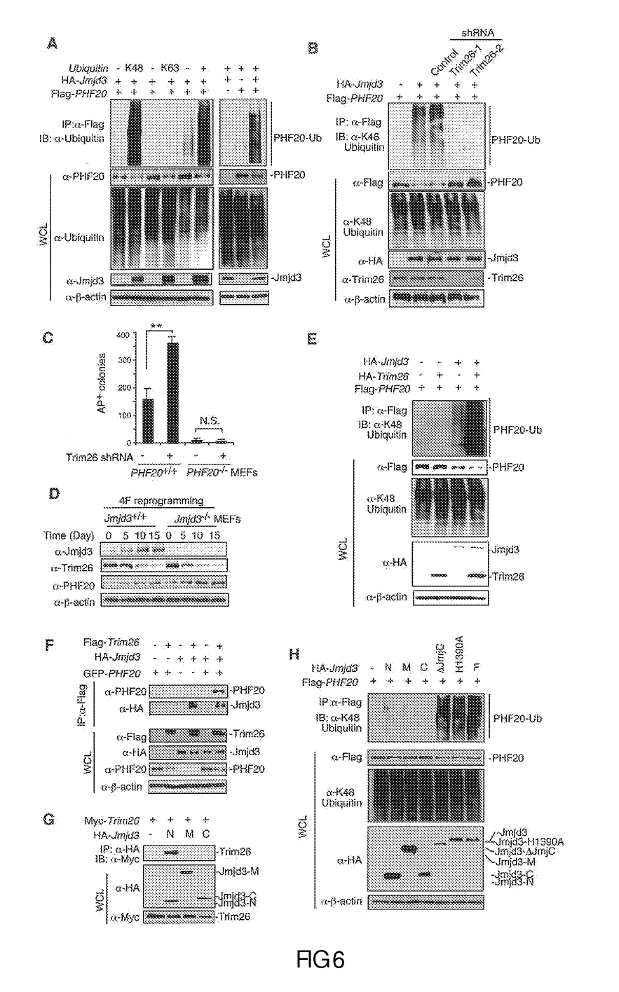

To dissect the molecular mechanisms by which Jmjd3 and PHF20 reciprocally control reprogramming, their subcellular distribution was first studied by immunofluorescent staining, with localization being observed in the nucleus. Fractionation of ESCs and iPSCs also confirmed this result. Coimmunoprecipitation (co-IP) and western blot analyses of 293T cells transfected with Flag-PHF20 and HA-Jmjd3 revealed that Jmjd3 interacted with PHF20 (FIG. 5A). Similar results were obtained with WT MEFs, but not with PHF20-deficient MEFs (FIG. 5B), suggesting that Jmjd3 interacts with PHF20 under physiological conditions. Domain-mapping experiments were then performed with Jmjd3-N (1-450 aa), Jmjd3-M (400-1200 aa) and Jmjd3-C (1201-1683 aa), which showed that the Jnmjd3-N and Jmjd3-C constructs, but not Jmjd3-M, interacted with PHF20 (FIG. 5C). Similarly, the N-terminal region (1-332 as containing a DNA binding domain), but not the C-terminal region, of PHF20 interacted with Jmjd3 (FIG. 5D). Further experiments showed that Jmjd3, but not Utx or Uty, interacted with PHF20. Thus, Jmjd3 specifically interacted with PHF20 via their functional domains.

What are the functional consequences of the Jmjd3-PHF20 interaction? To address this question, 293T cells were transfected with a fixed amount of Flag-PHF20 together with increasing amounts of HA-Jmjd3. In these studies, the amounts of PHF20 protein decreased with increasing expression of Jmjd3 protein. Similarly, the amounts of endogenous PHF20 protein were decreased in 293T cells transfected with increasing amounts of Jmjd3 cDNA (FIG. 5E). In support of this observation, the amount of endogenous PHF20 protein in Jmjd3 deficient MEFs was much higher than in WT MEFs, while ectopic expression of Jmjd3 cDNA in Jmjd3-deficient MEFs reduced the amount of PHF20 protein to a level similar to that in WT MEFs (FIG. 5F). It appears therefore, that Jmjd3 negatively regulates PHF20 protein by targeting it for degradation.

Trim26 is an E3 Ubiquitin Ligase Required for PHF20 Ubiquitination and Degradation

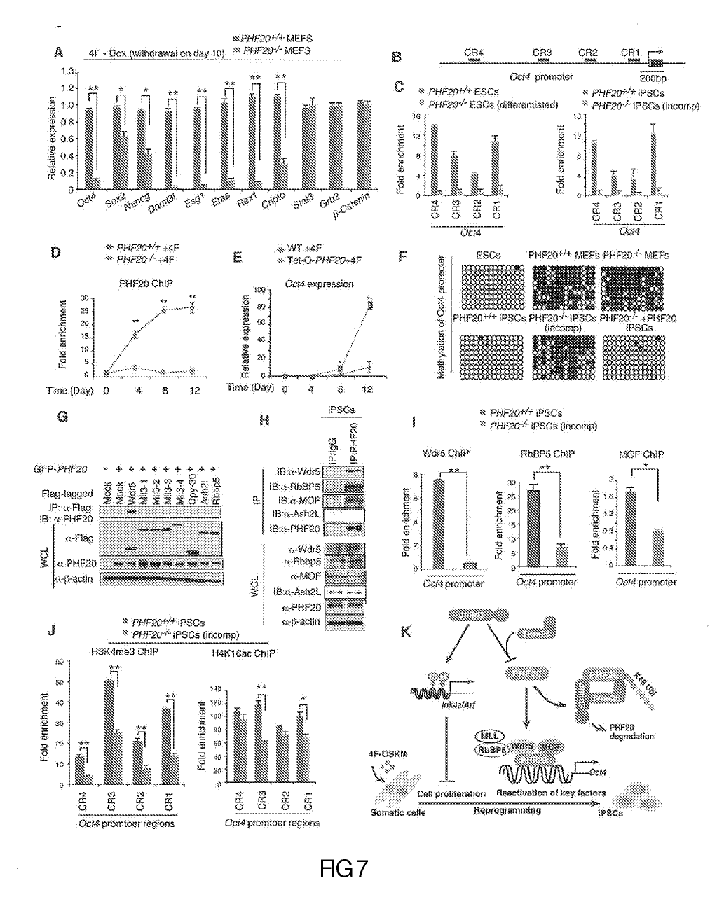

To determine how Jmjd3 causes the degradation of PHF20, the inventors first tested whether this protein was ubiquitinated in 293T cells expressing WT, K48 or K63 ubiquitin. PHF20 strongly underwent K48-linked ubiquitination, with little or no K63-linked ubiquitination, and such an ubiquitination was observed only when Jmjd3 and PHF20 were coexpressed in 293T cells (FIG. 6A). These results suggest that Jmjd3 specifically targets PHF20 for K48-linked polyubiquitination and proteasomal degradation.

Since Jmjd3 is not an E3 ubiquitin ligase, it was reasoned that Jmjd3 might function as an adaptor to recruit an E3 ubiquitin ligase to PHF20 for ubiquitination. To test this prediction, a screen was designed using 293T cells transfected with Jmjd3 expression vector and lentivirus-based shRNA constructs from a sublibrary of shRNAs for human E3 ubiquitin ligases, as previously described (Cui et al., 2012). In an initial screening of about 600 shRNAs, an E3 ubiqtuitin ligase (Trim26)-specific shRNA was identified that was associated with increased PHF20 protein amounts, relative to results with control shRNA. To substantiate this finding, two shRNAs were selected that showed 60% knockdown efficiency for human Trim26, and three murine Trim26-specific shRNAs with more than 90% knockdown efficiency. Knockdown of endogenous Trim26 by shRNAs markedly abrogated Jmjd3-mediated ubiquitination of PHF20 in 293T cells (FIG. 6B), with similar results obtained when either Jmjd3 or Trim26 was knocked down in PHF20⁴¹⁺ MEFs. Consistent with these results, it was found that knockdown of Trim26 increased reprogramming efficiency in PHF20 WT MEFs, but not in PHF20-deficient MEFs (FIG. 6C). Further studies showed that knockdown of Trim26 reversed Jmjd3-mediated inhibition of reprogramming while overexpression of Trim26 inhibited reprogramming efficiency enhanced by Jmjd3 knockdown.

Because Trim26 and Jmjd3 could act in concert to modulate reprogramming through targeting PHF20 for ubiquitination and degradation, the inventors next determined their expression patterns during reprogramming, and found that Trim26 was decreased while Jmjd3 was increased FIG. 6D). As expected, PHF20 expression gradually increased during reprogramming, but exhibited at a higher level in Jmjd3-1-MEFs than in WT MEFs (FIG. 6C and FIG. 6D). Although treatment with the protease inhibitor MG132 blocked protein degradation, and increased the amounts of PHF20 protein, even when both Trim26 and Jmjd3 were overexpressed, it was non-specific and caused cell death. Thus, no iPSC colony formation was observed after MG132 treatment. Taken together, these data indicated that the amounts of PHF20 protein. Jmjd3-Trim26 is responsible for the enhanced reprogramming efficiency observed in Jmjd3-1-MEFs or in Dox-treated Tet-O-PHF20 MEFs.

It was also determined that ectopic expression of Trim26 promoted PHF20 ubiquitination and degradation. Coexpression of Trim26 and Jmjd3 led to a remarkable increase in K48-linked ubiquitination and degradation of PHF20, compared with Trim26 or Jmjd3 alone (FIG. 6E). To determine whether Trim26 interacts with Jmjd3 or PHF20, immunoprecipitation experiments were performed using cells that expressed Jmjd3 alone, Trim26 alone, or PHF20 and Trim26 together. Although Trim26 interacted with Jmjd3 and not PHF20 (FIG. 6F), both Jmjd3 and PHF20 were detected in the anti-Flag-Trim26 immunoprecipitants of the cells that expressed Jmjd3, PHF20 and Trim26 (FIG. 6F), suggesting that Jmjd3 is an adaptor protein that recruits Trim26 to PHF20. To determine which domain of Jmjd3 is responsible for recruiting Trim26 to PHF20, 293T cells were transfected with Jmjd3-N, Jmjd3-M or Jmjd3-C together with Trim26. Immunoprecipitation and western blot experiments revealed that the N-terminus of Jmjd3 (Jmjd3-N), but not Jmjd3-M and Jmjd3-C, was capable of binding to Trim26 (FIG. 6G). To identify the domain of Jmjd3 that is required for Trim26-mediated ubiquitination of PHF20, 293T cells were transfected with Flag-PHF20 together with HA-tagged Jmjd3-N, Jmjd3-M, Jmjd3-C, Jmjd3-AlmjC, Jmjd3-H1390A, or full-length Jmjd3. After immunoprecipitation with anti-Flag, K48-linked ubiquitination of PHF20 was assessed. In this study it was observed that none of the Jmjd3-N, Jmjd3-M and Jmjd3-C constructs was sufficient to cause PHF20 ubiquitination (FIG. 6H). By contrast, like full-length Jmjd3. Jmjd3-41mjC and Jmjd3-H1390A were able to mediate PHF20 ubiquitination (FIG. 6H), consistent with results showing that Jmjd3-41mjC and Jmjd3-H1390A could still inhibit iPSC reprogramming in Jmjd3-deficient MEFs (FIG. 3F). Taken together, these results suggest that the N-terminus of Jmjd3 (Jmjd3-N) can interact with Trim26, but is not sufficient to cause PHF20 ubiquitination. Jmjd3 containing the first 1200 as or a point mutation (Jmjd3-6,3mjC or Jmjd3-H1390A) is necessary and sufficient to target PHF20 for ubiquitination by recruiting the E3 ligase Trim26.

PHF20 is Required for Endogenous Oct4 Expression and Interacts with WdrS During Reprogramming

Since PHF20 is essential for reprogramming in both WT and Jmjd3-deficient MEFs, we reasoned that it might be required for the reactivation of endogenous key genes such as Oct4 and other markers of ESCs. To test this prediction, we examined the effects of PHF20 loss on the activation of 11 ESC markers during Tet-O-4F-mediated reprogramming, using WT and PHF20ⁿ MEFs in both the presence of Dox and after its withdrawn Dox on day 10. Real-time PCR analysis on day 14 revealed that expression of Oct4, Sox2, Nanog, Dnmt31, Esgl, Eras, Rex1, and Cripto could not be activated or substantially reduced in PHF20-deficient MEFs, but were highly activated in WT MEFs even after withdrawal of Dox on day 10, while Stat3, Grb2 and bcatenin were activated normally in both WT and PHF20-deficient cells (FIG. 7A). Notably, Sox2 and Nanog could be reactivated when Dox was retained during reprogramming. Overexpression of Oct4 or even 4F could not rescue the incompletely reprogrammed phenotype of PHF20-deficient MEFs after reprogramming. These results strongly suggest that PHF20 is an upstream factor that controls many key reprogramming and pluripotency factors.

Because reactivation of endogenous Oct4 is essential for the generation of completely reprogrammed iPSCs (Ang et al., 2011), we next determined whether PHF20 could directly bind to the Oct4 promoter in vivo. ChIP-PCR assay revealed that PHF20 was strongly bound to this promoter in WT ESCs and iPSCs, but not in PHF20-deficient (differentiated) ESCs and (incompletely reprogrammed) iPSCs (FIG. 7B and FIG. 7C). PHF20 was unable to bind to the promoter regions of Cripto, Dnmt31, Esgl, Ems, Nonog, Rexl or Sox2. Consistently, ChIP-Seq analysis of ESCs and iPSCs further confirmed that PHF20 was bound to the Oct4 promoter, but was not able to bind to Sox2 or Nonog. Furthermore, the binding of PHF20 to the Oct4 promoter increased gradually over the course of reprogramming (FIG. 7D). To further determine whether overexpression of PHF20 could promote expression of endogenous Oct4, both WT and Tet-O-PHF20 MEFs expressing rtTA were treated with Dox during 4F-mediated reprogramming. The expression level of Oct4 was markedly increased in Dox-treated Tet-O-PHF20 MEFs, compared with Dox-treated rtTA-expressing WT MEFs (FIG. 7E), suggesting that PHF20 is required for the reactivation of endogenous Oct4 gene expression during reprogramming.

Because the DNA methylation status of the Oct4 promoter serves as an important marker of fully reprogrammed iPSCs (Stadtfeld and Hochedlinger, 2010), bisulfate sequencing analysis was performed for ESCs and iPSCs generated from WT MEFs, which showed robust DNA demethylation in the Oct4 promoter regions. By contrast, incompletely reprogrammed iPSC colonies from PHF20-deficient MEFs retained their DNA methylation pattern (FIG. 7F). More importantly, it was shown that ectopic expression of PHF20 could rescue the incompletely, reprogrammed state of PHF20-deficient iPSCs and the status of the Oct4 promoter demethylation, similar to results for WT iPSCs (FIG. 7F).

PHF20 is a component of mixed-lineage leukemia (MLL) H3K34 methyltransferase complexes with the core components MLL, ASH2L, WDR5 and RBBP5, as well as a component of the H4K16 acetyltransferase MOF (male-absent on the first, also called MYSTI, KAT8) complex (Cai et al., 2010; Dou et al., 2005; Mendjan et al., 2006; Wysocka et al., 2005). Importantly, Wdr5 is also a key component shared by MLL H3K4 methyltransferase and the H4K16 acet ltransferase MOP Cai et al., 2010; Dou et al., 2005; Mendan et al., 2006; W socka et al., 2005). However, it is not known whether PHF20 interacts with Wdr5 or other components of these two complexes. Because PHF20 is upregulated and binds to the Oct4 promoter during reprogramming, it was predicted that PHF20 might interact with Wdr5 to promote endogenous Oct4 expression during reprogramming. To test this possibility, 293T cells were transfected with PHF20 together with Wdr5, MLL3, Dpv-30, Ash21 or RhBP5, all core components of the H3K4 methyltransferase complex (Trievel and Shilatifard, 2009). PHF20 interacted with Wdr5, but neither with MLL3, Dpy-30, Ash21, RbBP5 (FIG. 7G), nor with key reprogramming factors Oct4, Sox2 or Nanog. Endogenous interactions between PHF20 and Wdr5 or RbBP5 (but not Ash2L) were observed in iPSCs (FIG. 7H). ChIP-seq analysis of ESCs and iPSCs confirmed that both PHF20 and Wdr5 were bound to the Oct4 promoter. Among 4830 genes bound by PHF20 and 5320 genes by Wdr5, approximately 1900 genes were co-occupied by PHF20 and Wdr5. Co-immunoprecipitation with anti-PHF20 revealed that PHF20 interacted with endogenous MOF in iPSCs (FIG. 7H), consistent with the results of a recent report showing that H3K4 methylation is closely associated with H4K16 acetylation (Wang et al., 2009). Thus, PHF20 interacted with Wdr5 and MOF to bring the H3K4 methyltransferase complex and H4K16 aceyltransferase MOF complex together.

To understand how the loss of PHF20 results in failure to reactivate endogenous Oct4 expression, the possibility that PHF20 might affect the ability of Wdr5, RbBP5 and MOF to bind to the Oct4 promoter region was examined. In ChIP-PCR experiments with WT and PHF20-deficient cells, Wdr5 failed to bind to the Oct4 promoter in PHF20-deficient cells, but bound strongly to the Oct4 promoter in WT cells (FIG. 7I). Similarly, the ability of RbBP5 and MOF to bind to the Oct4 promoter was markedly reduced in PHF20-deficient cells. Consistent with these results, ChIP-qPCR experiments revealed a sharp reduction in H3K4 trimethylation in the Oct4 promoter, while H4K16 acetylation was also affected but to a lesser extent (FIG. 7J). Taken together, these results suggested that binding of PHF20 to the Oct4 promoter may be required for recruiting 1J3K4 methyltransferase complex and perhaps H4K16 acetlytransferase complex to bind to the same promoter through the interaction with Wdr5 and MOF, leading to reactivation of endogenous Oct4 expression during reprogramming (FIG. 7K).

### REFERENCES

1. Adams-Cioaba, M A et al., FEBS Lett., 586:859-865 (2012).
2. Agger. K et al., Genes Dev., 23:1171-1176 (2009).
3. Agger. K et al., Nature, 449:731-734 (2007).
4. Ang, Y S et al., Cell, 145:183-197 (2011).
5. Badeaux, Al et al., J. Biol. Chem., 287:429-437 (2012).
6. Barradas, M et al., Genes Dev., 23:1177-1182 (2009).
7. Brahmer, J R et al., N. Engl. J. Med., 366:2455-2465 (2012).
8. Buonerba, C et al., Expert Rev. Anticancer Ther., 11:25-28 (2011).
9. Cal, Y et al., J. Biol. Chem., 285:4268-4272 (2010).
10. Carey, B W et al., Nat. Methods, 7:56-59 (2010).
11. Chen, S et al., Genes Dev., 26:1364-1375 (2012).
12. Chen, Y T et al., PLoS One 6, e17876 (2011).
13. Cui, J et al., Cell, 141:483-496 (2010).
14. Cui, J et al., Nat. Immunol., 13:387-395 (2012).
15. Curiel, T J et al., Nat. Med., 10:942-949 (2004).
16. Darce, J et al., Immunity, 36:731-741 (2012).
17. Davis, I D et al., Proc. Natl. Acad. Sci. USA, 101:10697-10702 (2004).
18. Di Lorenzo, G et al., Nat. Rev. Clin. Oncol., 8:551-561 (2011).
19. Dong, H et al., Nat. Med., 5:1365-1369 (1999).
20. Dong, H et al., Nat. Med., 8:793-800 (2002).
21. Dou. Y et al., Cell, 121:873-885 (2005).
22. Ea, C K et al., Mol. Cell, 22:245-257 (2006).
23. Ezhkova, E et al., Cell, 136:1122-1135 (2009).
24. Ferrari. M Nat. Rev. Cancer, 5:161-171 (2005).
25. Ferrari, M Nat. Rev. Clin. Oncol., 7:485-486 (2010).
26. Fischer, U et al., Clin. Exp. Immunol., 126:206-213 (2001).
27. Fischer, U et al., Clin. Exp. Immunol., 126:206-213 (2001).
28. Fu, T et al., J. Clin. Invest., 114:542-550 (2004).
29. Gaspar-Maia, A et al., Nature, 460:863-868 (2009).
30. Gaspar-Maia, A et al., Nat. Rev. Mol. Cell Biol., 12:36-47 (2011).
31. Grattoni, A et al., Pharm. Res., 28:292-300 (2011).
32. Hanna, J et al., Nature, 462:595-601 (2009).
33. Hemberger, M et al., Nat. Rev. Mol. Cell Biol., 10:526-537 (2009).
34. Hochedlinger, K and Plath, K, Development, 136:509-523 (2009).
35. Hodi, F S et al., N. Engl. J. Med. 363:711-723 (2010).
36. Hong, H et al., Nature, 460:1132-1135 (2009).
37. Hong, S et al., Proc. Natl. Acad. Sci. USA, 104:18439-18444 (2007).
38. Jepsen, K et al., Nature, 450:415-419 (2007).
39. Kantoff, P W et al., New Engl. J. Med., 363:411-422 (2010).
40. Kawamura, T et al., Nature, 460:1140-1144 (2009).
41. Khong, H T et al., J. Immunother., 27:472-477 (2004).
42. Kim, J et al., EMBO Rep., 7:397-403 (2006).
43. Koche, R P et al., Cell Stem Cell, 8:96-105 (2011).
44. Kouzarides, T Cell. 128:693-705 (2007).
45. Lan, F et al., Nature, 449:689-694 (2007).
46. Langmead, B et al., Genome Biol., 10:R25 (2009).
47. Lesterhuis, W J et al., Nat. Rev. Drug Discov., 10:591-600 (2011).
48. Li, H et al., Nature, 460:1136-1139 (2009).
49. Li, R et al., Cell Stem Cell, 7:51-63 (2010).
50. Li, X et al., Cell Stem Cell, 11:163-178 (2012a).
51. Li, Y et al., Cell Signal, 25:74-84 (2012b).
52. Liu, T et al. Genome Biol., 12:R83 (2011).
53. Loh, Y H et al., Genes Dev., 21:2545-2557 (2007).
54. Mansour, A A et al., Nature, 488:409-413 (2012).
55. Marion, R M et al., Nature, 460:1149-1153 (2009).
56. Matsuzaki, J et al., Proc. Natl. Acad. Sci. USA. 107:7875-7880 (2010).
57. Matys, V et al., Nucl. Acids Res., 34:D108-110 (2006).
58. Mendjan, S et al., Mol. Cell, 21:811-823 (2006).
59. Miller, S A et al., Mol. Cell, 40:594-605 (2010).
60. Moore, C B et al., Nature, 451:573-577 (2008).
61. Odunsi, K et al., Proc. Natl. Acad. Sci. USA, 104:12837-12842 (2007).
62. Okita, K et al., Nature, 448:313-317 (2007).
63. Onder. T T et al., Nature. 483:598-602 (2012).
64. Pallasch, C P et al., Int. J. Cancer, 117:456-459 (2005).
65. Papp, B and Plath, K Cell Res., 21:486-501 (2011).
66. Park, I H et al., Nat. Protocol., 3:1180-1186 (2008).
67. Park, S et al., J. Biol. Chem., 287:11151-11163 (2012).
68. Peng, G et al., Science, 309:1380-1384 (2005).
69. Plath, K and Lowry, W E Nat. Rev. Genet., 12:253-265 (2011).
70. Robinton, D A and Daley, G Q Nature, 481:295-305 (2012).
71. Rosenberg, S A, Nat. Rev. Clin. Oncol., 8:577-585 (2011).
72. Sakaguchi, S Annu. Rev. Immunol., 22:531-562 (2004).
73. Samstein, R M et al., Cell, 150:29-38 (2012).
74. Samstein, R M et al., Cell, 151:153-166 (2012).
75. Shin, H et al., Bioinformatics, 25:2605-2606 (2009).
76. Singhal, N et al., Cell, 141:943-955 (2010).
77. Smith, Z D et al., Nat. Biotechnol., 28:521-526 (2010).
78. Song, X T et al., Nat. Med., 14:258-265 (2008).
79. Stadtfeld, M and Hochedlinger, K Genes Dev., 24:2239-2263 (2010).
80. Stadtfeld, M et al., Nat. Methods, 7:53-55 (2010).
81. Su, I H et al., Nat. Immunol., 4:124-131 (2003).
82. Takahashi, K and Yamanaka, S Cell, 126:663-676 (2006).
83. Takahashi, K et al., Cell, 131:861-872 (2007b).
84. Takahashi, K et al., Nat. Protocol., 2:3081-3089 (2007a).
85. Tanaka, T et al., Cancer Res., 70:3687-3696 (2010).
86. Tasciotti, E et al., Nat. Nanotechnol., 3:151-157 (2008).
87. Taube, J M et al., Sci. Transl. Med., 4:127ra137 (2012).
88. Tong, Y et al., Cell Res., 22:822-835 (2012).
89. Topalian, S L et al., N. Engl. J. Med., 366:2443-2454 (2012).
90. Trievel, R C and Shilatifard, A Nat. Struct. Mol. Biol., 16:678-680 (2009).
91. Utikal, J et al., Nature, 460:1145-1148 (2009).
92. Valmori, D et al., Proc. Natl. Acad. Sci. USA, 104:8947-8952 (2007).
93. Valouev, A et al., Nat. Methods, 5:829-834 (2008).
94. Wang, H Y and Wang, R F Curr. Opin. Immunol., 19:217-223 (2007).
95. Wang, H Y et al., J. Clin. Investig., 109:1463-1470 (2002).
96. Wang, H Y et al., Immunity. 20:107-118 (2004).
97. Wang, L et al., Bioinformatics, 26:136-138 (2010).
98. Wang, T et al., Cell Stem Cell, 9:575-587 (2011).
99. Wang, Y et al., J. Immunol., 169:1102-1109 (2002).
100. Wang, Z et al., Cell, 138:1019-1031 (2009).
101. Wernig. M et al., Nat. Biotechnol., 26:916-924 (2008).
102. Woo, S R et al., Cancer Res., 72:917-927 (2012).
103. Wysocka, J et al., Cell, 121:859-872 (2005).
104. Xia, X et al., Immunity, 34:843-853 (2011).
105. Yu, J et al., Science, 318:1917-1920 (2007).
106. Zeng, G et al., J. Immunol., 165:1153-1159 (2000).
107. Zou, W Nat. Rev. Immunol., 6:295-307 (2006).
108. Zou. W and Chen, L Nat. Rev. Immunol., 8:467-477 (2008).

1. A pharmaceutical composition comprising a PHF20 peptide which is derived from the PHF20 protein and is a cytotoxic T lymphocyte (CTL) epitope, and a pharmaceutically acceptable excipient.

2. The pharmaceutical composition of claim 1, wherein the PHF20 peptide is able to stimulate T cells so that the T cells are able to recognize T2 cells loaded with a PHF20 peptide, or PHF20-positive breast cancer cells.

3. The pharmaceutical composition of claim 1, wherein the PHF20 protein from which a PHF20 peptide is derived is the human PHF20 protein.

4. The pharmaceutical composition of claim 1, wherein the PHF20 peptide comprises a amino acid sequence selected from the group consisting of SEQ ID NOs: 1 to 10.

5. The pharmaceutical composition of claim 1, wherein the PHF20 peptide comprises the amino acid sequence WQFNLLTHV (SEQ ID NO: 2).

6. The pharmaceutical composition of claim 1, further comprising an adjuvant.

7. A method for treating, preventing, or managing breast cancer in a subject in need thereof comprising administering to said subject the pharmaceutical composition of claim 1.

8. The method of claim 7, further comprising administering to the subject an immune response modifier.

9. The method of claim 7, wherein negative regulators of dendritic cells in the patient is knocked down.

10. The method of claim 7, wherein anti-PD-1 blockade is induced in the patient, concurrently or after the administration of the pharmaceutical composition.

11. The method according to claim 10, wherein an effective amount of an anti-PD-1 siRNA is administered to the patient.

12. The method according to claim 10, where an effective amount of an anti-PD-1 antibody is administered to the patient.

13. The method of claim 7, wherein the subject is human.

14. A method according to claim 7, wherein a subject in need thereof is administered a PHF20 peptide loaded on a dendritic cell.

15. A method according to claim 7, wherein the pharmaceutical composition is administered to the patient under conditions sufficient for the patient to develop a cytotoxic T-Cell (CTL) response.

16. (canceled)

17. (canceled)

18. A method of treating breast cancer, comprising (a) isolating a cell population containing or capable of producing CTLs and/or T_{H} cells from a subject; (b) treating the cell population with a PHF20 peptide, optionally together with a proliferative agent; (c) screening the cell population for CTLs or T_{H} cells or their combination, with specificity to a PHD20 peptide; and (d) administering the cell population to a patient suffering from cancer.

19. A method of treating breast cancer according to claim 18, the method further comprising: (d) cloning T cell receptor (TCR) genes from the screened CTLs, T_{H} cells or their combination with specificity to the PHF20 peptide described herein; (e) transducing the TCR gene cloned in step (c) into either: i. cells from the patient; or ii. cells from a donor; or iii. eukaryotic or prokaryotic cells for the generation of monoclonal TCRs (mTCRs); and (f) administering the transduced cells or generated mTCRs from step (e) to a patient suffering from breast cancer.

20. A method for treating, preventing, or managing breast cancer in a subject in need thereof comprising administering to said subject a pharmaceutical composition comprising an antibody against PHF20.

21. The method according to claim 20, wherein the antibody is a humanized antibody, or a human antibody, or a monoclonal antibody.

22. The method according to claim 7, further comprising administering to said subject a pharmaceutical composition comprising an antibody against PHF20.

23. The method according to claim 10, further comprising administering to said subject a pharmaceutical composition comprising an antibody against PHF20.

24. (canceled)
